SEQUENCE LISTING (39 sequences)

SEQ ID NO: 1 - DNA, 23 bp, Unknown
  Q445F, forward primer for a xenotropic murine leukemia virus related virus (XMRV) gag G1 sequence used in the quantitative real time polymerase chain reaction study of XMRV
ggactttttggagtggctttgtt

SEQ ID NO: 2 - DNA, 21 bp, Unknown
  Q538R, reverse primer for a xenotropic murine leukemia virus related virus (XMRV) gag G1 sequence used in the quantitative real time polymerase chain reaction study of XMRV
gcgtaaaaccgaaagcaaaat

SEQ ID NO: 3 - DNA, 22 bp, Unknown
  480F, probe used in the quantitative real time polymerase chain reaction study of XMRV
acagagacacttcccgcccccg

SEQ ID NO: 4 - DNA, 25 bp, Unknown
  625F, forward primerfor a xenotropic murine leukemia virus related virus (XMRV) gag G2 sequence used in the quantitative real time polymerase chain reaction study of XMRV
gtaactacccctctgagtctaacct

SEQ ID NO: 5 - DNA, 24 bp, Unknown
  708R, reverse primer for a xenotropic murine leukemia virus related virus (XMRV) gag G2 sequence used in the quantitative real time polymerase chain reaction study of XMRV
cttcttgacatccacagactggtt

SEQ ID NO: 6 - DNA, 16 bp, Unknown
  668F, probe used in the quantitative real time polymerase chain reaction study of XMRV
tccagcgcattgcatc

SEQ ID NO: 7 - DNA, 26 bp, Unknown
  797F, forward primer for a xenotropic murine leukemia virus related virus (XMRV) gag G3 sequence used in the quantitative real time polymerase chain reaction study of XMRV
ctcaggtcaagtctagagtgttttgt

SEQ ID NO: 8 - DNA, 22 bp, Unknown
  874R, reverse primer for a xenotropic murine leukemia virus related virus (XMRV) gag G3 sequence used in the quantitative real time polymerase chain reaction study of XMRV
cctcccaggtgacgatatatgg

SEQ ID NO: 9 - DNA, 14 bp, Unknown
  834F, probe used in the quantitative real time polymerase chain reaction study of XMRV
ccccacggacaccc

SEQ ID NO: 10 - DNA, 25 bp, Unknown
  4843F, forward primer for a xenotropic murine leukemia virus related virus (XMRV) gag pol P1 sequence used in the quantitative real time polymerase chain reaction study of XMRV
cgggacagaactatccagtatgtga

SEQ ID NO: 11 - DNA, 22 bp, Unknown
  4912R, reverse primer for a xenotropic murine leukemia virus related virus (XMRV) gag pol P1 sequence used in the quantitative real time polymerase chain reaction study of XMRV
tggctttgctggcatttacttg

SEQ ID NO: 12 - DNA, 16 bp, Unknown
  4873F, probe used in the quantitative real time polymerase chain reaction study of XMRV
acctgcaccgcctgtg

SEQ ID NO: 13 - DNA, 18 bp, Unknown
  6124F, forward primer for a xenotropic murine leukemia virus related virus (XMRV) gag env E1 sequence used in the quantitative real time polymerase chain reaction study of XMRV
ggccgagagagggctact

SEQ ID NO: 14 - DNA, 24 bp, Unknown
  6197R, reverse primer for a xenotropic murine leukemia virus related virus (XMRV) gag env E1 sequence used in the quantitative real time polymerase chain reaction study of XMRV
tgatgatgatggcttccagtatgc

SEQ ID NO: 15 - DNA, 16 bp, Unknown
  6159R, probe used in the quantitative real time polymerase chain reaction study of XMRV
cacatccccatttgcc

SEQ ID NO: 16 - DNA, 20 bp, Unknown
  ENV-F, forward primer for a xenotropic murine leukemia virus related virus (XMRV) gag env E2 sequence used in the quantitative real time polymerase chain reaction study of XMRV
ccctagtggccaccaaacaa

SEQ ID NO: 17 - DNA, 21 bp, Unknown
  ENV-R, reverse primer for a xenotropic murine leukemia virus related virus (XMRV) gag env E2 sequence used in the quantitative real time polymerase chain reaction study of XMRV
aaggccccaaggtctgtatgt

SEQ ID NO: 18 - DNA, 21 bp, Unknown
  7192F, probe used in the quantitative real time polymerase chain reaction study of XMRV
tcgagcagctccaggcagcca

SEQ ID NO: 19 - DNA, 21 bp, Unknown
  ENV-F, forward primer for a xenotropic murine leukemia virus related virus (XMRV) gag env E3 sequence used in the quantitative real time polymerase chain reaction study of XMRV
tcaggacaagggtggtttgag

SEQ ID NO: 20 - DNA, 21 bp, Unknown
  ENV-R, reverse primer for a xenotropic murine leukemia virus related virus (XMRV) gag env E3 sequence used in the quantitative real time polymerase chain reaction study of XMRV
ggcccataatggtggatatca

SEQ ID NO: 21 - DNA, 27 bp, Unknown
  7480, probe used in the quantitative real time polymerase chain reaction study of XMRV
ttaacaggtccccatggttcacgacca

SEQ ID NO: 22 - DNA, 28 bp, Unknown
  6200R, reverse primer used in the Tth-nested real time polymerase chain reaction for xenotropic murine virus (XMRV)
cccatgatgatgatggcttccagtatgc

SEQ ID NO: 23 - DNA, 22 bp, Unknown
  5922F, forward primer used in the Tth-nested real time polymerase chain reaction for xenotropic murine virus (XMRV)
gctaatgctacctccctcctgg

SEQ ID NO: 24 - DNA, 23 bp, Unknown
  350F, forward primer used in the Tth-nested real time polymerase chain reaction for xenotropic murine virus (XMRV)
gagttcgtattcccggccgcagc

SEQ ID NO: 25 - DNA, 28 bp, Unknown
  718R, reverse primer used in the Tth-nested real time polymerase chain reaction for xenotropic murine virus (XMRV)
ggtaacccagcgcctcttcttgacatcc

SEQ ID NO: 26 - DNA, 23 bp, Unknown
  5942F, forward primer used in the Tth-nested real time polymerase chain reaction for xenotropic murine virus (XMRV)
ggggacgatgacagacactttcc

SEQ ID NO: 27 - DNA, 23 bp, Unknown
  6159R, reverse primer used in the Tth-nested real time polymerase chain reaction for xenotropic murine virus (XMRV)
cacatccccatttgccacagtag

SEQ ID NO: 28 - DNA, 25 bp, Unknown
  424F, forward primer used in the Tth-nested real time polymerase chain reaction for xenotropic murine virus (XMRV)
atcagttaacctacccgagtcggac

SEQ ID NO: 29 - DNA, 23 bp, Unknown
  535R, reverse primer used in the Tth-nested real time polymerase chain reaction for xenotropic murine virus (XMRV)
ggtttcggcgtaaaaccgaaagc

SEQ ID NO: 30 - DNA, 26 bp, Unknown
  6273R, reverse primer used in the Tth-nested real time polymerase chain reaction for xenotropic murine virus (XMRV)
ggagcccactgaggaatcaaaacagg

SEQ ID NO: 31 - DNA, 22 bp, Unknown
  5922F, forward primer used in the Tth-nested real time polymerase chain reaction for xenotropic murine virus (XMRV)
gctaatgctacctccctcctgg

SEQ ID NO: 32 - DNA, 23 bp, Unknown
  350F, forward primer used in the Tth-nested real time polymerase chain reaction for xenotropic murine virus (XMRV)
gagttcgtattcccggccgcagc

SEQ ID NO: 33 - DNA, 28 bp, Unknown
  718R, reverse primer used in the Tth-nested real time polymerase chain reaction for xenotropic murine virus (XMRV)
ggtaacccagcgcctcttcttgacatcc

SEQ ID NO: 34 - DNA, 38 bp, Unknown
  a 112 nts band amplified with forward primer 424 and reverse primer 535R
atcagttaacctacccgagtcggactttttggagtggc

SEQ ID NO: 35 - DNA, 36 bp, Unknown
  a 112 nts band amplified with forward primer 424 and reverse primer 535R
tttgttgggggacgagagacagagacacttcccgcc

SEQ ID NO: 36 - DNA, 38 bp, Unknown
  a 112 nts band amplified with forward primer 424 and reverse primer 535R
cccgtctgaatttttgctttcggttttacgccgaaacc

SEQ ID NO: 37 - DNA, 80 bp, Unknown
  a 218 nts band amplified with forward primer 5942F and reverse primer 6159R
ggggacgatgacagacactttccctaaactatattttgacttgtgtgatttagttggagacaactgggatgacccggaac

SEQ ID NO: 38 - DNA, 80 bp, Unknown
  a 218 nts band amplified with forward primer 5942F and reverse primer 6159R
ccgatattggagatggttgccgctctcccgggggaagaaaaaggacaagactatatgatttctatgtttgccccggtcat

SEQ ID NO: 39 - DNA, 58 bp, Unknown
  a 218 nts band amplified with forward primer 5942F and reverse primer 6159R
actgtattaacagggtgtggagggccgagagagggctactgtggcaaatggggatgtg

(19) United States
(12) Patent Application Publication
(10) Pub. No.: US 2010/0184015 A1
(43) Pub. Date: Jul. 22, 2010

(54) METHOD FOR DETECTION OF XMRV

(76) Inventors: Robert H. Silverman, Beachwood, OH (US); Eric A. Klein, Shaker Heights, OH (US); Christopher J. Weight, Shaker Heights, OH (US); Carvell T. Nguyen, University Heights, OH (US); Jaydip Das Gupta, Mayfield Heights, OH (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/645,181

(22) Filed: Dec. 22, 2009

Related U.S. Application Data
(60) Provisional application No. 61/203,556, filed on Dec. 23, 2008.

(51) Int. Cl. C12Q 1/70

(52) U.S. Cl. 435/5

(57) ABSTRACT

The present invention relates to the identification of Xenotropic murine leukemia virus (XMRV) nucleic acid by polymerase chain reaction (PCR) analysis (e.g., real time PCR (RT/PCR); nested RT/PCR using Tth DNA polymerase and Hot start polymerase) and the uses thereof. In particular, the invention provides methods for the detection, and in particular early detection, of XMRV in RNA isolated from samples (e.g., urine samples; expressed prostate secretion (EPS)) of prostate cancer patients and normal individuals.

### RELATED APPLICATION(S)

This application claims the benefit of U.S. Provisional Application No. 61/203,556, filed on Dec. 23, 2008. The entire teachings of the above application(s) are incorporated herein by reference.

### GOVERNMENT SUPPORT

The invention was supported, in whole or in part, by a grant W81XWH-07-1-0338 from the Department of Defense Prostate Cancer research program (PCRP). The Government has certain rights in the invention.

### BACKGROUND OF THE INVENTION

Prostate cancer is the leading cause of non-cutaneous malignancies and the second leading cause of cancer-related deaths among American men. A need exists for improved methods of detection, particularly early detection, of prostate cancer.

### SUMMARY OF THE INVENTION

The present invention relates to the identification of Xenotropic murine leukemia virus (MLV) related virus (XMRV) nucleic acid by polymerase chain reaction (PCR) analysis (e.g., real time PCR (RT/PCR); nested RT/PCR using Tth DNA polymerase and Hot start polymerase) and the uses thereof. In particular, the invention provides methods for the detection, and in particular early detection, of XMRV nucleic acic (e.g., RNA, DNA) in samples (e.g., urine samples; expressed prostate secretion (EPS), blood, semen, seminal vesicle fluids or the like) of prostate cancer patients and normal individuals.

In one aspect, the invention is directed to a method of detecting the presence of xenotropic MLV related virus (XMRV) in an individual. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence, or a combination thereof. The sample is maintained under conditions which amplify the primers if XMRV is present in the sample to produce amplified XMRV sequences. Whether amplified XMRV sequences are present in the sample are detected, wherein if amplified XMRV sequences are detected in the sample, then XMRV is present in the individual.

In another aspect, the invention is directed to method of detecting prostate cancer (e.g., at an early stage) in an individual. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence, or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates that the individual has prostate cancer at an early stage.

The invention also provides a method of detecting an individual at risk for developing prostate cancer. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence, or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates that the individual is at risk for developing prostate cancer.

The invention also provides a method of detecting recurrence of prostate cancer in an individual. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pot nucleotide sequence, or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates the recurrence of prostate cancer in the individual.

The invention also provides a method of monitoring a treatment of an individual that has prostate cancer. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence; or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates that the treatment is likely not effective or is likely not yet effective.

### BRIEF DESCRIPTION OF THE DRAWINGS

FIG. 1 is a schematic diagram of the gag (G1, G2, G3), poi (P1) and env (E1, E2, E3) regions of XMRV.

FIGS. 2A and 2B are standard curves of envelope RNA using E3 (FIG. 2A) and G3 (FIG. 2B) which was diluted to different dilutions and analyzed by qRT/PCR using Ag-Path kit.

FIGS. 3A and 3B show the results of XMRV RNA copy number in urine of prostate cancer patient VP663 using E1 site in env and E2 site in env, respectively. Results are shown of qRT-PCR assays performed six times (x) and in comparison to a standard curve generated with a 1.85 kb XMRV env RNA produced by in vitro transcription. Y axis shows the Ct values, x axis shows the log of the copy number.

FIG. 4 shows the results of detection of XMRV RNA in EPS of prostate cancer patient VP 657 and VP 635 using E1 site in env. Results of qRT-PCR assays were preformed in duplicate and are shown in comparison to a standard curve generated with a 1.85 kb XMRV env RNA produced by in vitro transcription. Y axis shows the Ct values, x axis shows the log of the copy number.

FIG. 5 shows the amplification plot of qRT/PCR identification of XMRV RNA in prostatitis patient using the E1 primer-probe combination. Duplicate samples were assayed which shows very high Ct value corresponding to very low copy number.

FIG. 6A shows an amplification plot of qRT/PCR analysis of XMRV RNA in prostate cancer patient's EPS (pj 339) using the G2 primer-probe combination.

Similarly the assay shown in FIG. 6B shows an amplification plot of prostate cancer patient EPS (pj 301, 302 and 304) using the E1 primer probe combination.

FIG. 7, upper panel, provides a schematic diagram showing the regions used in the nested RT/PCR analysis. The lower panel of FIG. 7 is an agarose gel showing the detection of XMRV RNA isolated from an XMRV infected prostate cancer cell line and RNA from prostate cancer patient EPS (pj 339) generated bands of 218 and 112 nucleotides in length.

FIG. 8 shows a 2% agarose gel of the nested RT-PCR product of RNA samples isolated from 6 prostate cancer patients' urine samples using Tth polymease for RT and first round PCR, followed by Taq DNA Polymerase for second round PCR amplification.

FIG. 9 shows a 2% agarose gel of the nested RT-PCR product of RNA samples isolated from 17 prostate cancer patients' expressed prostate secretion (EPS) during prostatectomy using Tth polymerase for RT and first round PCR, followed by Taq DNA Polymerase for second round PCR amplification.

FIG. 10 shows the sequences of the bands of 112 (SEQ ID NOs: 34, 35 and 36) and 218 (SEQ ID NOs: 37, 38 and 39) nucleotides in length referred to in FIG. 7.

FIG. 11 is a gel of singleplex nested RT-PCR of RNA isolated from 3 prostate cancer patient urine samples, reaction time were done in triplicates. Oligos 6200R and 5922F were used for the first round followed by 6159R and 5942F for the second round of amplification.

FIG. 12 is a graph showing the detection and determination of XMRV DNA copy numbers in DNA isolated from tumor-bearing prostate tissues of men with the RNASEL QQ genotype following prostatectomy.

### DETAILED DESCRIPTION OF THE INVENTION

Hereditary prostate cancer (HPC), which accounts for 43% of early onset cases and about 9% of all cases, is due to germline mutations in HPC genes (Carter, B. S., et al., Proc. Natl. Acad. Sci. USA, 89(8):3367 (1992)). In 2002, the first HPC gene was reported (Carpten, J., et al., Nat. Genet., 30(2):181 (2002)). HPC1 encodes RNase L, an essential protein in antiviral innate immunity (reviewed in Silverman, R., Cytkine Growth Factor Rev., 18(5-6):381 (2007)). Genetic evidence that an antiviral gene suppresses prostate cancer led to examination of the possibility that chronic viral infections might predispose men to prostate cancer. In 2006 discovery of a new human retrovirus, xenotropic MLV related virus (XMRV), in tumor-bearing prostate tissues, was reported (Urisman, A., et al., PLoS Pathog., 2(3):e25 (2006)). Remarkably, XMRV is present in prostate tissues of men that are homozygous for a reduced activity variant of RNase L, but rarely in men with wild type RNase L. In 2007, construction of an infectious viral molecular clone of XMRV was reported (Dong, B., et al., Proc. natl. Acad. Sci., USA, 104(5):1655 (2007)). Methods of monitoring XMRV infections as an indicator or predictor of prostate cancer progression or aggressiveness are provided herein.

Specifically, described herein is the development of polymerase chain reaction (PCR) assays (e.g., real-time quantitative RT-PCR (qRT-PCR) assays) for the detection of XMRV nucleic acid in a sample (e.g., urine and other bodily fluids, such as prostate secretions, and semen) obtained from an individual (e.g., patient). In particular aspects, highly sensitive, specific and quantitative real-time (RT) PCR assays for XMRV nucleic acid (e.g., DNA; RNA) and nested RT-PCR assays for detection of XMRV nucleic acid are described. These assays are useful for determination of viral loads in tissues and fluids from individuals with and without cancer. Also described herein is the correlation of the prevalence and load of XMRV in prostate cancer cases with disease parameters. XMRV is a newly discovered infection of tumor-bearing prostate that correlates with mutations in a prostate cancer susceptibility gene (RNASEL). The occurrence of XMRV infections in prostate cancer cases provides for pathogenesis of the disease, assessing risk, and novel therapeutic options.

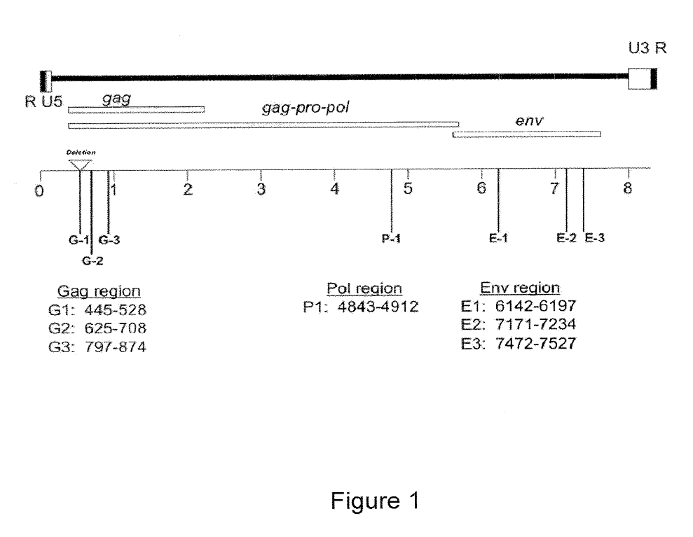

In one aspect, total RNA was extracted from prostate tissue samples using Trizol reagent (Invitrogen, Carlsbad, Calif.) and from urine and prostatic secretion using the Magmax™ Viral RNA Isolation Kit (Ambion Inc., Austin Tex.) and then stored at −80° C. until further processing. Initial screening of samples to detect XMRV gene sequences was performed using qRT-PCR (performed on an Applied Biosystems 7500 Real Time PCR system). In one embodiment, these reactions are performed using a one-step RT-PCR reaction (AgPath-ID™ kit, Applied Biosystems). PCR assays for seven different regions in XMRV RNA have been developed (FIG. 1) which involved the design of sets of Taqman-based primers/probe used to detect three regions in XMRV RNA, including one region of gag (G1) and two regions of env (E1 & E2).

The presence of XMRV in prostatic secretions and urine as shown herein is significant because such data provides a prostatic secretion- and urine-based XMRV detection assay that is non-invasive, rapid, and easy to perform, avoiding the morbidity and difficulty of obtaining blood or tissue specimens for sampling. Current screening for prostate cancer by prostate-specific antigen (PSA) levels and digital rectal exam often does not begin until age 50 and has significant limitations and inaccuracies. In contrast to these tests, the assays for XMRV described herein can be performed on much younger men, especially those with a family history of prostate cancer. Because men with XMRV infections, especially those that fail to clear the virus, are likely at increased risk of prostate cancer, these studies provide a new diagnostic for evaluating risk of prostate cancer initiation or progression.

Accordingly, in one aspect, the invention is directed to a method of detecting the presence of xenotropic MLV related virus (XMRV) in an individual.

As used herein, “XMRV” refers to an infectious gammaretrovirus found in prostate tumors, particularly in prostate tumors of patients homozygous for RNASEL variant, R462Q (e.g., Urisman, A., et al., PLoS Pathog., 2(3):e25 (2006); Dong, B., et al., Proc. Natl. Acad. Sci., USA, 104(5):1655 (2007); and WO 2006/110589; all of which are incorporated herein by reference in their entirety). The term “XMRV” includes any strain of the virus including XMRV VP35 (GenBank Accession No. DQ241301), XMRV VP42 (GenBank Accession No. DQ241302) and XMRV VP62 (GenBank Accession No. DQ399707).

As used herein an “individual” refers to any subject in need of screening. In particular embodiments, the individual is a mammal, such as a primate (e.g., human), cow, sheep, goat, horse, dog, cat, rabbit, guinea pig, rat, mouse or other bovine, ovine, equine, canine feline, rodent or murine species). In one embodiment, the individual is a human. In another embodiment, the individual is a human under the age of 50 years, 40 years, 30 years or 20 years. In another embodiment, the individual is a cancer patient (e.g., a prostate cancer patient; and HPC patient). In another embodiment, the individual is in remission from prostate cancer. In another embodiment, the individual has or has had a (one or more) XMRV infection. In another embodiment, the individual's genome comprises a wild type, a heterozygous or a homozygous mutation of the RNase L gene. In yet another embodiment, the individual expresses a mutated or variant form of RNase L (e.g., R462Q; QQ RNASEL).

Although the invention is performed herein using urine and/or prostatic secretion samples so as to demonstrate that the method is non-invasive, rapid and easy to perform, one of skill in the art will appreciate that any suitable biological sample obtained from an individual can be used in the methods of the invention. The sample can be a biological fluid, a tissue sample (e.g., prostate, bladder, seminal glands, testes, kidney, bone marrow, colon, ileum, jejunum, pancreas, adrenal glands, liver, heart, lung, spleen, brain cortex, brain stem, cerebellum, inguinal lymph node, axillar lymph node and mesenteric lymph node), a tumor sample (e.g., a prostate tumor, a bladder tumor, other tumors of the male and female genitourinary tracts) and combinations thereof. A suitable sample can be obtained for example by cell or tissue biopsy. A sample can also be obtained from other tissues, bodily fluids and products, e.g., from a tissue smear, tissue scrape, and the like. Thus, the sample can be a biopsy specimen (e.g, tumor, polyp, mass (solid, cellular)), aspirate, and/or smear sample). The sample can be from a tissue that has a tumor (e.g., cancerous growth) and/or tumor cells, or is suspected of having a tumor and/or tumor cells. For example, a tumor biopsy can be obtained in an open biopsy, a procedure in which an entire (excisional biopsy) or partial (incisional biopsy) mass is removed from a target area. Alternatively, a tumor sample can be obtained through a percutaneous biopsy, a procedure performed with a needle-like instrument through a small incision or puncture (with or without the aid of a imaging device) to obtain individual cells or clusters of cells (e.g., a fine needle aspiration (FNA)) or a core or fragment of tissues (core biopsy).

In a particular embodiment, the sample is a biological fluid. Examples of a biological fluid that can be used in the methods include urine, prostatic fluids, blood and semen. As used herein, “prostatic fluids” include expressed prostate secretions (EPS) such as semen.

As described herein, the sample is contacted with at least one set of primers and maintained under conditions which amplify the primers if XMRV is present in the sample to produce amplified XMRV nucleic acid sequences, also referred to herein as “amplicons” or “XMRV amplicons”. The amplified XMRV nucleic acid sequences can be, for example, XMRV DNA or XMRV RNA.

A “set of primers” comprises at least one forward primer and at least one reverse primer, wherein the forward primer and the reverse primer in the set are complementary to all or a portion of an XMRV nucleotide sequence (e.g., XMRV G1, XMRV G2, XMRV G3, XMRV P1, XMRV E1, XMRV E2, XMRV E3). Typically, the forward primer and the reverse primer within a set of primers are complementary to all or a portion of the same region or a similar region of the XMRV nucleotide sequence (e.g., the gag region, the env region, the pol region). As used herein, the term “primer” refers to an oligonucleotide, which is capable of acting as a point for the initiation of synthesis of a primer extension product that is complementary to a target nucleotide sequence that is to be amplified, referred to as the target or template nucleic acid sequence. In this instance, the target or template nucleic acid sequence is all or a portion (e.g., the gag region, the env region, the pol region) of an XMRV nucleic acid sequence. The primer may occur naturally, as in a purified restriction digest, or be produced synthetically. The appropriate length of a primer depends on the intended use of the primer, but typically ranges from about 5 to about 100; from about 5 to about 75; from about 5 to about 50; from about 5 to about 10; from about 10 to about 35; from about 18 to about 22 nucleotides. A primer need not reflect the exact sequence of the target sequence but must be sufficiently complementary to hybridize with the target sequence for primer elongation to occur, i.e., the primer is sufficiently complementary to the target nucleotide sequence such that the primer will anneal to the template under conditions that permit primer extension. Reverse transcription can be performed with M-MLV RT (such as Superscript™ 1, II or III (Invitrogen)) or Tth DNA polymerase in RT buffer using Oligo dT, random hexamer or XMRV gene specific primers. As used herein, the phrase “conditions that permit primer extension” refers to those conditions, e.g., salt concentration (metallic and non-metallic salts), pH, temperature, and necessary cofactor concentration, among others, under which a given polymerase enzyme catalyzes the extension of an annealed primer. Conditions for the primer extension activity of a wide range of polymerase enzymes are known in the art. As one example, conditions permitting the extension of a nucleic acid primer by Taq polymerase include the following (for any given enzyme, there can and often will be more than one set of such conditions): reactions are conducted in a buffer containing 50 mM KCl, 10 mM Tris (pH 8.3-8.6), 1.5-4 mM MgCl₂, 200 μM of dNTPs; reactions can be performed at about 68°-72° C.

It will be clear to persons skilled in the art that the size of the primer and the stability of hybridization will be dependent to some degree on the ratio of A-T to C-G base pairings, since more hydrogen bonding is available in a C-G pairing. Also, the skilled person will consider the degree of homology between the extension primer to other parts of the amplified sequence and choose the degree of stringency accordingly. Guidance for such routine experimentation can be found in the literature, for example, Molecular Cloning: a laboratory manual by Sambrook, J., Fritsch E. F. and Maniatis, T. (1989) which is incorporated herein by reference.

In addition to the primer pairs, probes can be included with reporter dye at the 5′ end (e.g., fluorescein, 6-carboxy fluorescein (FAM), 6-FAM, 5-FAM, TAMRA) and quencher dye at the 3′ end (e.g., BHQ-1, BHQ-2, TAMRA, MGB) which will bind to the XMRV DNA during PCR (e.g., U.S. Pat. No. 7,374,833 which is incorporated herein by reference).

For detection purposes, the primer can comprises at least one tag or label. As used herein, “tag” or “label” are used interchangeably to refer to any moiety that is capable of being specifically detected (e.g., by a partner moiety), either directly or indirectly, and therefore, can be used to identify and/or isolate a polynucleotide sequence that comprises the tag. Suitable tags for the present invention include, among others, affinity tags (e.g., biotin, avidin, streptavidin), haptens, ligands, peptides, nucleic acids, fluorophores, chromophores, and epitope tags that are recognized by an antibody (e.g., digoxigenin (DIG), hemagglutinin (HA), myc, Flag) (Andrus, A. “Chemical methods for 5′ non-isotopic labelling of PCR probes and primers” (1995) in PCR 2: A Practical Approach, Oxford University Press, Oxford, pp. 39-54). Other suitable tags include, but are not limited to, chromophores, fluorophores, haptens, radionuclides (e.g., ³²P, ³³P, ³⁵S), fluorescence quenchers, enzymes, enzyme substrates, affinity tags (e.g., biotin, avidin, streptavidin, etc.), mass tags, electrophoretic tags and epitope tags that are recognized by an antibody. In certain embodiments, the label is present on the 5 carbon position of a pyrimidine base or on the 3 carbon deaza position of a purine base.

The primers have a nucleotide sequence that is complementary to all or a portion of an XMRV sequence. In particular embodiments, the primers have a nucleotide sequence that is complementary to all or a portion of an XMRV gag sequence, an XMRV pol, an XMRV env sequence or a combination thereof.

In one embodiment, at least one forward primer and at least one reverse primer are complementary to all or a portion of an XMRV G1 gag nucleotide sequence. As used herein, an “XMRV G1 gag nucleotide sequence” refers to a sequence that is from about nucleotide 445 to about nucleotide 528 of an XMRV genomic sequence. The length of the probe which binds between two primers in the G1 gag nucleotide sequence can vary between about 12 to about 40 nucleotides complementary to all or a portion of XMRV G1 gag nucleotide sequence. Minor groove binding principle can also be applied when the probe size is as short as about 12 nucleotides. In a particular embodiment, the forward primer complementary to all or a portion of an XMRV G1 gag nucleotide sequence has a nucleotide sequence comprising GGACTTTTTGGAGTGGCTTTGTT (SEQ ID NO: 1), the reverse primer complementary to all or a portion of an XMRV G1 gag nucleotide sequence has a nucleotide sequence comprising GCGTAAAACCGAAAGCAAAAT (SEQ ID NO: 2) and the probe has a nucleotide sequence comprising ACAGAGACACTTCCCGCCCCCG (SEQ ID NO: 3).

In another embodiment, at least one forward primer and at least one reverse primer are complementary to all or a portion of an XMRV G2 gag nucleotide sequence. As used herein, an “XMRV G2 gag nucleotide sequence” refers to a sequence that is from about nucleotide 625 to about nucleotide 708 of an XMRV genomic sequence. The length of probe which binds between two primers in the G2 gag nucleotide sequence can vary between about 12 to about 40 nucleotides complementary to all or a portion of XMRV G1 gag nucleotide sequence. Minor groove binding can also be applied when the probe size is as short as about 12 nucleotides. In a particular embodiment, the forward primer complementary to all or a portion of an XMRV G2 gag nucleotide sequence has a nucleotide sequence comprising GTAACTACCCCTCTGAGTCTAACCT (SEQ ID NO: 4), the reverse primer complementary to all or a portion of an XMRV G3 gag nucleotide sequence has a nucleotide sequence comprising CTTCTTGACATCCACAGACTGGTT (SEQ ID NO: 5) and the probe has a nucleotide sequence comprising TCCAGCGCATTGCATC (SEQ ID NO: 6).

In another embodiment, at least one forward primer and at least one reverse primer are complementary to all or a portion of an XMRV G3 gag nucleotide sequence. As used herein, an “XMRV G3 gag nucleotide sequence” refers to a sequence that is from about nucleotide 797 to about nucleotide 874 of an XMRV genomic sequence. The length of the probe which binds between two primers in the G3 gag nucleotide sequence can vary between about 12 to about 40 nucleotides complementary to all or a portion of XMRV G1 gag nucleotide sequence. Minor groove binding principle can also be applied when the probe size is as short as about 12 nucleotides. In a particular embodiment, the forward primer complementary to all or a portion of an XMRV G3 gag nucleotide sequence has a nucleotide sequence comprising CTCAGGTCAAGTCTAGAGTGTTTTGT (SEQ ID NO: 7), the reverse primer complementary to all or a portion of an XMRV G2 gag nucleotide sequence has a nucleotide sequence comprising CCTCCCAGGTGACGATATATGG (SEQ ID NO: 8) and the probe has a nucleotide sequence comprising CCCCACGGACACCC (SEQ ID NO: 9).

In another embodiment, at least one forward primer and at least one reverse primer are complementary to all or a portion of an XMRV P1 Pol nucleotide sequence. As used herein, an “XMRV P1 Pol nucleotide sequence” refers to a sequence that is from about nucleotide 4843 to about nucleotide 4912 of an XMRV genomic sequence. The length of the probe which binds between two primers in the P1 pol nucleotide sequence can vary between about 12 to about 40 nucleotides complementary to all or a portion of XMRV G1 gag nucleotide sequence. Minor groove binding principle can also be applied when the probe size is as short as about 12 nucleotides. In a particular embodiment, the forward primer complementary to all or a portion of an XMRV P1 pol nucleotide sequence has a nucleotide sequence comprising CGGGACAGAACTATCCAGTATGTGA (SEQ ID NO: 10), the reverse primer complementary to all or a portion of an XMRV P1 pol nucleotide sequence has a nucleotide sequence comprising TGGCTTTGCTGGCATTTACTTG (SEQ ID NO: 11) and the probe has a nucleotide sequence comprising ACCTGCACCGCCTGTG (SEQ ID NO: 12).

In another embodiment, at least one forward primer and at least one reverse primer are complementary to all or a portion of an XMRV E1 env nucleotide sequence. As used herein, an “XMRV E1 env nucleotide sequence” refers to a sequence that is from about nucleotide 6142 to about nucleotide 6197 of an XMRV genomic sequence. The length of the probe which binds between two primers in the E1 env nucleotide sequence can vary between about 12 to about 40 nucleotides complementary to all or a portion of XMRV G1 gag nucleotide sequence. Minor groove binding principle can also be applied when the probe size is as short as about 12 nucleotides. In a particular embodiment, the forward primer complementary to all or a portion of an XMRV E1 env nucleotide sequence has a nucleotide sequence comprising GGCCGAGAGAGGGCTACT (SEQ ID NO: 13), the reverse primer complementary to all or a portion of an XMRV E1 env nucleotide sequence has a nucleotide sequence comprising TGATGATGATGGCTTCCAGTATGC (SEQ ID NO: 14) and the probe has a nucleotide sequence comprising CACATCCCCATTTGCC (SEQ ID NO: 15).

In another embodiment, at least one forward primer and at least one reverse primer are complementary to all or a portion of an XMRV E2 env nucleotide sequence. As used herein, an “XMRV E2 env nucleotide sequence” refers to a sequence that is from about nucleotide 7171 to about nucleotide 7234 of an XMRV genomic sequence. The length of the probe which binds between two primers in the E2 env nucleotide sequence can vary between about 12 to about 40 nucleotides complementary to all or a portion of XMRV G1 gag nucleotide sequence. Minor groove binding principle can also be applied when the probe size is as short as about 12 nucleotides. In a particular embodiment, the forward primer complementary to all or a portion of an XMRV E2 env nucleotide sequence has a nucleotide sequence comprising CCCTAGTGGCCACCAAACAA (SEQ ID NO: 16), the reverse primer complementary to all or a portion of an XMRV E2 env nucleotide sequence has a nucleotide sequence comprising AAGGCCCCAAGGTCTGTATGT (SEQ ID NO: 17) and the probe has a nucleotide sequence comprising TCGAGCAGCTCCAGGCAGCCA (SEQ ID NO: 18).

In another embodiment, at least one forward primer and at least one reverse primer are complementary to all or a portion of an XMRV E3 env nucleotide sequence. As used herein, an “XMRV E3 env nucleotide sequence” refers to a sequence that is from about nucleotide 7472 to about nucleotide 7527 of an XMRV genomic sequence. The length of the probe which binds between two primers in the E3 env nucleotide sequence can vary between about 12 to about 40 nucleotides complementary to all or a portion of XMRV G1 gag nucleotide sequence. Minor groove binding principle can also be applied when the probe size is as short as about 12 nucleotides. In a particular embodiment, the forward primer complementary to all or a portion of an XMRV E3 env nucleotide sequence has a nucleotide sequence comprising TCAGGACAAGGGTGGTTTGAG (SEQ ID NO: 19), the reverse primer complementary to all or a portion of an XMRV E3 env nucleotide sequence has a nucleotide sequence comprising GGCCCATAATGGTGGATATCA (SEQ ID NO: 20) and the probe has a nucleotide sequence comprising TTAACAGGTCCCCATGGTTCACGACCA (SEQ ID NO: 21).

The primers are amplified using any suitable method known in the art. As used herein, “amplification” or an “amplification reaction” refers to any suitable method for amplification of a nucleic acid sequence including polymerase chain reaction (PCR), ligase chain reaction (LCR), rolling circle amplification (RCA), strand displacement amplification (SDA) and multiple displacement amplification (MDA), as will be understood by a person of skill in the art. Such methods for amplification typically comprise, e.g., primers that anneal to the nucleic acid sequence to be amplified, a DNA polymerase, and nucleotides. Furthermore, amplification methods, such as PCR, can be solid-phase amplification, polony amplification, colony amplification, emulsion PCR, bead RCA, surface RCA, surface SDA, etc., as will be recognized by one of skill in the art. It will also be recognized that it is advantageous to use an amplification method that results in exponential amplification of free DNA molecules in solution or tethered to a suitable matrix by only one end of the DNA molecule. In addition, it will be recognized that it is often advantageous to use amplification protocols that maximize the fidelity of the amplified products to be used as templates in DNA sequencing procedures. Such protocols use, for example, DNA polymerases with strong discrimination against misincorporation of incorrect nucleotides and/or strong 3′ exonuclease activities (also referred to as proofreading or editing activities) to remove misincorporated nucleotides during polymerization.

In one embodiment, a PCR method is used to amplify the primers. As known to those of skill in the art, PCR is a technique in which a DNA polymerase is used to amplify a piece of DNA (e.g., a gene or portion thereof; a non-coding region) by in vitro enzymatic replication. As PCR progresses, the DNA generated is used as a template for replication which sets in motion a reaction in which the DNA template is exponentially amplified. With PCR a single or few copies of a piece of DNA are amplified across several orders of magnitude, generating millions or more copies of the DNA piece. As is also known in the art, PCR can be extensively modified to perform a wide array of genetic manipulations.

PCR applications typically employ a heat-stable (thermostabile) polymerase (e.g., DNA polymerase). A variety of polymerases for use in PCR are known to this of skill in the art and include Taq polymerase, an enzyme originally isolated from the bacterium Thermus aquaticus, and Vent and Tth polymerases derived from microorganisms that normally reside at high temperature. Consequently, these polymerase enzymes are quite stable to heat denaturation, making them ideal enzymes for use in the polymerase chain reaction. These polymerases, such as a DNA polymerase enzymatically assembles a new DNA strand from DNA building blocks, the nucleotides, by using single-stranded DNA as a template and DNA oligonucleotides (also called DNA primers), which are required for initiation of DNA synthesis.

PCR methods typically use thermal cycling, i.e., alternately heating and cooling the PCR sample to a defined series of temperature steps. These thermal cycling steps physically separate the strands (at high temperatures) in a e.g., DNA double helix (DNA melting) used as the template during DNA synthesis (at lower temperatures) by the DNA polymerase to selectively amplify the target DNA. Selectivity of PCR arises from the use of primers that are complementary to the DNA region targeted for amplification under specific thermal cycling conditions.

PCR typically involves the use of several components and reagents such as a nucleic acid (e.g., DNA) template that contains the region (target) to be amplified; one or more, typically two or more, primers which are complementary to the nucleic acid regions at the 5′ (five prime) or 3′ (three prime) ends of the nucleic acid region; one or more polymerases e.g., with a temperature optimum at around 70° C.; one or more deoxynucleoside triphosphates (dNTPs; also very commonly and erroneously called deoxynucleotide triphosphates), the building blocks from which the DNA polymerases synthesizes a new DNA strand; one or more buffer solutions, providing a suitable chemical environment for optimum activity and stability of the polymerase; one or more divalent cations, e.g., magnesium or manganese ions; generally Mg2+ is used, but Mn2+ can be utilized for PCR-mediated DNA mutagenesis, as higher Mn2+ concentration increases the error rate during DNA synthesis; and one or more monovalent cation potassium ions.

PCR is commonly carried out in a reaction volume of 10-200 μl in small reaction tubes (0.2-0.5 ml volumes) in a thermal cycler which heats and cools the reaction tubes to achieve the temperatures required at each step of the reaction. Although one of skill in the art will appreciate that PCR can occur in a variety of ways depending upon the desired result(s), an example of a PCR can occur as follows. The PCR can begin with an initialization step, which involves heating the reaction to a temperature of about 94-96° C. (or about 98° C. if extremely thermostable polymerases are used), which is held for about 1-9 minutes. This is typically used with DNA polymerases that require heat activation by hot-start PCR. A denaturation step, which is the first regular cycling event, involves heating the reaction to about 94-98° C. for about 20-30 seconds. This results in melting of DNA template and primers by disrupting the hydrogen bonds between complementary bases of the DNA strands, yielding single strands of DNA. An annealing step, which involves lowering the temperature to about 50-65° C. for about 20-40 seconds allowing annealing of the primers to the single-stranded DNA template, can then be carried out. Typically the annealing temperature is about 3-5 degrees Celsius below the melting temperature (Tm) of the primers used. Stable DNA-DNA hydrogen bonds are generally formed when the primer sequence very closely matches the template sequence. The polymerase binds to the primer-template hybrid and begins DNA synthesis.

In the extension/elongation step, the temperature depends on the DNA polymerase used; Taq polymerase has its optimum activity temperature at about 75-80° C., and commonly a temperature of about 72° C. is used with this enzyme. At this step the DNA polymerase synthesizes a new DNA strand complementary to the DNA template strand by adding dNTPs that are complementary to the template in 5′ to 3′ direction, condensing the 5′-phosphate group of the dNTPs with the 3′-hydroxyl group at the end of the nascent (extending) DNA strand. The extension time depends both on the DNA polymerase used and on the length of the DNA fragment to be amplified. A final elongation step is occasionally performed at a temperature of about 70-74° C. for about 5-15 minutes after the last PCR cycle to ensure that any remaining single-stranded DNA is fully extended. A final hold step at about 4-15° C. for an indefinite time can be employed for short-term storage of the reaction.

In a particular embodiment, a real time (RT/PCR) or quantitative, real time PCR (qRT/PCR) reaction is used to amplify the primers if XMRV is present in the sample. As understood by one of skill in the art, RT/PCR DNA simultaneously quantifies and amplifies the nucleic acid. In this method, the nucleic acid is specifically amplified by polymerase chain reaction. After each round of amplification, the DNA is quantified. Common methods of quantification include the use of fluorescent dyes that intercalate with double-strand nucleic acid and modified oligonucleotides (called probes) that fluoresce when hybridized with a complementary DNA.

Specifically, quantitative PCR (Q-PCR) is used to measure the quantity of a PCR product (preferably real-time). The method quantitatively measures starting amounts of DNA, cDNA or RNA. Q-PCR is commonly used to determine whether a DNA sequence is present in a sample and the number of its copies in the sample, and is also known as RT-PCR (Real Time PCR), RQ-PCR, QRT-PCR or RTQ-PCR. RT-PCR commonly refers to reverse transcription PCR, which can also be used in the methods described herein, and is often used in conjunction with Q-PCR. QRT-PCR methods use fluorescent dyes, such as Sybr Green, or fluorophore-containing DNA probes, such as TaqMan, to measure the amount of amplified product in real time.

Real-time polymerase chain reaction, also called quantitative real time polymerase chain reaction (Q-PCR/qPCR) or kinetic polymerase chain reaction, is based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA.

Frequently, real-time polymerase chain reaction is combined with reverse transcription polymerase chain reaction to quantify low abundance messenger RNA (mRNA), enabling one of skill in the art to quantify relative gene expression at a particular time, or in a particular cell or tissue type. Although real-time quantitative polymerase chain reaction is sometimes incorrectly abbreviated as RT-PCR, it should not be confused with reverse transcription polymerase chain reaction, also known as RT-PCR.

The reaction is typically run in a thermocycler as described herein, and after each cycle, the levels of fluorescence are measured with a detector; the dye only fluoresces when bound to the dsDNA (i.e., the PCR product). With reference to a standard dilution, the dsDNA concentration in the PCR can be determined.

A comparison of a measured DNA/RNA sample to a standard dilution provides a fraction or ratio of the sample relative to the standard, allowing relative comparisons between different tissues or experimental conditions. The method can further comprise normalizing expression of a target gene to a stably expressed gene.

In another embodiment, fluorescent reporter probes are used. A sequence-specific RNA and/or DNA-based probe is used to quantify the nucleic acid containing the probe sequence; therefore, use of the reporter probe can increase specificity, and allow quantification even in the presence of some non-specific DNA amplification. This allows for multiplexing—assaying for several genes in the same reaction by using specific probes with different-coloured labels, provided that all genes are amplified with similar efficiency.

The reaction is typically carried out with an RNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the polymerase (e.g., taq polymerase) breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter.

The PCR is prepared as usual, and the reporter probe is added. As the reaction commences, during the annealing stage of the PCR both probe and primers anneal to the DNA target. Polymerisation of a new DNA strand is initiated from the primers, and once the polymerase reaches the probe, its 5′-3-exonuclease degrades the probe, physically separating the fluorescent reporter from the quencher, resulting in an increase in fluorescence. Fluorescence is detected and measured in the real-time PCR thermocycler, and its geometric increase corresponding to exponential increase of the product is used to determine the threshold cycle (CT; Ct) in each reaction.

In intact probes, reporter fluorescence is quenched. Probes and the complementary DNA strand are hybridized and reporter fluorescence is still quenched. During PCR, the probe is degraded by the polymerase and the fluorescent reporter released.

Relative concentrations of DNA present during the exponential phase of the reaction can be determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Ct. Since the quantity of DNA doubles every cycle during the exponential phase, relative amounts of DNA can be calculated, e.g. a sample whose Ct is 3 cycles earlier than another's has 23=8 times more template.

Amounts of RNA or DNA are then determined by comparing the results to a standard curve produced by real-time PCR of serial dilutions (e.g. undiluted, 1:4, 1:16, 1:64) of a known amount of RNA or DNA. As mentioned above, to quantify gene expression, the measured amount of RNA from the gene of interest is divided by the amount of RNA from a control sequence (also referred to herein as a reference or housekeeping sequence) (e.g., gene) measured in the same sample to normalize for possible variation in the amount and quality of RNA between different samples. This normalization permits accurate comparison of expression of the sequence of interest between different samples, provided that the expression of the reference (housekeeping) sequence used in the normalization is very similar across all the samples.

In another embodiment, nested, reverse transcription PCR is used to amplify the primers. Nested PCR is a PCR with a second round of amplification using a different set of primers. This second set of primers is specific to a sequence found within the nucleotide sequence of the initial conventional PCR amplicon. The use of a second amplification step with the “nested” primer set results in a reduced background from products amplified during the initial PCR due to the nested primers' additional specificity to the region. The amount of amplicon produced is increased as a result of the second round of amplification and due to a reduction in any inhibitor concentrations. Reverse transcription, nested PCR indicates that the reaction is initiated with DNA that has been reverse transcribed from RNA.

As described herein, whether amplified XMRV sequences are present in the sample are detected, wherein if amplified XMRV sequences are detected in the sample, then XMRV is present in the individual. Detection of amplified XMRV sequences can be achieved by resolving sequences by means of, for example, gel electrophoresis (e.g., agarose gel), high-resolution denaturing polyacrylamide/urea gel electrophoresis, capillary separation, or other resolving means; followed by detecting the sequence using, for example, a scanning spectrophotometer or fluorometer. In a particular embodiment, fluorescently-labeled amplified XMRV sequences are resolved by gel electrophoresis, according to procedures that are well known in the art, and are subsequently detected in the gel using a standard fluorometer. In one embodiment, a positive XMRV generates a band of 218 nucleotides in length, 112 nucleotides in length or a combination thereof.

The method can further comprise determining the sequences of the amplified XMRV sequence using procedures well known in the art.

As discussed above and as apparent to one of skill in the art, the method of detecting the presence of XMRV in a sample can further comprise the use of a control. That is, the amount or level of amplified XMRV nucleic acid sequences in the sample can be compared to the amount or level of amplified XMRV nucleic acid sequences in a control sample. Suitable controls are well recognized in the art and include, for example, a sample from an individual that is known to not be infected with XMRV, a sample from an individual that is known to be infected with XMRV, a sample from an individual that is a prostate cancer patient (e.g., HPC patient), and/or a reference standard of authentic (positive) XMRV RNA. The control sample can be the same type of sample as the sample obtained from the individual (e.g., the sample obtained from the individual and the control sample are urine samples) or the control sample can be a different sample (e.g., the sample obtained from the individual is a urine sample and the control sample is a tissue sample such as a prostate tissue sample).

The methods for detecting XMRV is an individual can be used for a variety of purposes such as for diagnostic and/or prognostic purposes for predicting (or indicating) a clinical outcome (e.g., relapse, metastasis, survival) of a newly diagnosed prostate cancer patient or a prostate cancer patient that is undergoing or has undergone therapy.

Accordingly, the invention is directed to method of detecting prostate cancer at an early stage in an individual. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence, or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates that the individual has prostate cancer at an early stage.

The invention also provides a method of detecting an individual at risk for developing prostate cancer. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pot nucleotide sequence, or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates that the individual is at risk for developing prostate cancer.

The invention also provides a method of detecting recurrence of prostate cancer in an individual. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence, or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates the recurrence of prostate cancer in the individual.

The invention also provides a method of monitoring a treatment of an individual that has prostate cancer. The method comprises contacting a sample of the individual with at least one set of primers wherein the set of primers comprises at least one forward primer and at least one reverse primer which are complementary to all or a portion of an XMRV G1 gag nucleotide sequence, at least one forward primer and at least one reverse primer which are complementary to all or a portion of an XMRV G2 gag nucleotide sequence, at least one forward primer and at least one reverse primer which are complementary to all or a portion of an XMRV G3 gag nucleotide sequence, at least one forward primer and at least one reverse primer which are complementary to all or a portion of an XMRV E1 envelope nucleotide sequence, at least one forward primer and at least one reverse primer which are complementary to all or a portion of an XMRV E2 envelope nucleotide sequence, at least one forward primer and at least one reverse primer which are complementary to all or a portion of an XMRV E3 envelope nucleotide sequence, at least one forward primer and at least one reverse primer which are complementary to all or a portion of an XMRV P1 Pol nucleotide sequence; or a combination thereof. The sample is maintained under conditions which amplify the primers if XMRV is present in the sample to produce amplified XMRV sequences, and whether amplified XMRV sequences are present in the sample are detected. The detection of amplified XMRV sequences in the sample indicates that the treatment is likely not effective or is likely not yet effective.

### Example 1

### Detection of XMRV RNA by qRT-PCR

### Methods

Primers and Probes Used in qRT/PCR Study of XMRV

Gag (G1):

(SEQ ID NO: 1)

| | |
|---|---|
| Q445T: | GGACTTTTTGGAGTGGCTTTGTT |

(SEQ ID NO: 2)

| | |
|---|---|
| Q528R: | GCGTAAAACCGAAAGCAAAAT |

(SEQ ID NO: 3)

| | |
|---|---|
| Probe (480F): | FAM/ACAGAGACACTTCCCGCCCCCG/BHQ1 |

Product size: 84 nts
Gag TaqMan ® MGB (G2):

(SEQ ID NO: 4)

| | |
|---|---|
| 625F: | GTAACTACCCCTCTGAGTCTAACCT |

(SEQ ID NO: 5)

| | |
|---|---|
| 708R: | CTTCTTGACATCCACAGACTGGTT |

(SEQ ID NO: 6)

| | |
|---|---|
| Probe (668E): | FAM/TCCAGCGCATTGCATC/MGB |

Product size: 82 nts
Gag TaqMan ® MGB (G3):

(SEQ ID NO: 7)

| | |
|---|---|
| 797F: | CTCAGGTCAAGTCTAGAGTGTTTTGT |

(SEQ ID NO: 8)

| | |
|---|---|
| 874R: | CCTCCCAGGTGACGATATATGG |

(SEQ ID NO: 9)

| | |
|---|---|
| Probe (834F): | FAM/CCCCACGGACACCC/MGB |

Product size: 78 nts
Pol TaqMan ® MGB (P1):

(SEQ ID NO: 10)

| | |
|---|---|
| 4843F: | CGGGACAGAACTATCCAGTATGTGA |

(SEQ ID NO: 11)

| | |
|---|---|
| 4912R: | TGGCTTTGCTGGCATTTACTTG |

(SEQ ID NO: 12)

| | |
|---|---|
| Probe (4873F): | FAM/ACCTGCACCGCCTGTG/MGB |

Product size: 70 nts
GP70 TaqMan ® MGB (E1):

(SEQ ID NO: 13)

| | |
|---|---|
| 6124F: | GGCCGAGAGAGGGCTACT |

(SEQ ID NO: 14)

| | |
|---|---|
| 6197R: | TGATGATGATGGCTTCCAGTATGC |

(SEQ ID NO: 15)

| | |
|---|---|
| Probe (6159R): | FAM/CACATCCCCATTTGCC/MGB |

Product size: 72 nts
P15E ENY (E2):

(SEQ ID NO: 16)

| | |
|---|---|
| ENV- F: | CCCTAGTGGCCACCAAACAA (7171E) |

(SEQ ID NO: 17)

| | |
|---|---|
| ENV- R: | AAGGCCCCAAGGTCTGTATGT (7234R) |

(SEQ ID NO: 18)

| | |
|---|---|
| Probe (7192F): | FAM/TCGAGCAGCTCCAGGCAGCCA/BHQ1 |

Product size: 64 nts
P15E Env (E3):

(SEQ ID NO: 19)

| | |
|---|---|
| ENV- F: | TCAGGACAAGGGTGGTTTGAG (7472F) |

(SEQ ID NO: 20)

| | |
|---|---|
| ENV- R: | GGCCCATAATGGTGGATATCA (7527R) |

(SEQ ID NO: 21)

| | |
|---|---|
| Probe (7480): | TAM/TTAACAGGTCCCCATGGTTCACGACCA/BHQ1 |

Product size: 56 nts

TaqMan® MGB (minor groove binder) primer probe combination is obtained as a premix format (25× concentration) from Applied Biosystems. For non-MOB primers and probes, the oligonucleotides were resuspended to a stock concentration of 100 uM (100 picomoles/ul) in 1×Tris-EDTA (TE) buffer. Aliquots of 50 uM of primers and 10 uM of probe working solution were made. The working probe was protected from light by covering with aluminum foil.

### Reagents

1. Trizol Reagent (Invitrogen)

2. MagMax™ Viral RNA isolation kit (Ambion Cat: AM 1939)

3. Non-stick RNase-Free 1.5 ml microfuge tubes (Ambion Cat: AM 12450)

4. RNA Storage solution (Ambion Cat: AM 7000)

5. AgPath-ID™ one-step RT/PCR Kit (Ambion Cat: AM 1005)

6. 1× TE buffer (USB Cat: 75893)

7. DEPC treated water (USB Cat: 70783)

8. PCR-Qualified water (USB Cat: 71875)

9. 96 well plate and optical adhesive cover (Applied Biosystems P/N 4311971).

Standard RNA and PCR precautions were used (e.g., used powder free gloves, filter tips and clean area for RNA and PCR work).

### Recommendations:

1. Freshly frozen prostate tissue was the best source for XMRV identification. Paraffin embedded tissue was not a good source.

2. During the prostatectomy in the operating room, the prostate juice was collected in RNase free labeled tubes and immediately frozen in dry ice.

3. Glass homogenizer were not used to mince the frozen prostate tissues to avoid cross contamination.

4. When possible, a separate space for dealing with prostate RNA was designated.

5. Preferably separate pipettes were used to add reagents, which were not used to pipette high copy standard plasmid or RNA.

6. Preferably, at least two replicates of qRT/PCR were performed on various patient RNA samples.

### RNA:

1. RNA was isolated from prostate tissue or from prostate secretion.

2. In vitro transcribed XMRV RNA (XMRV VP62 RNA sequence between nucleotides 5761 and 7691) was used for detection of Env RNA or in vitro transcribed XMRV RNA (XMRV VP62 RNA sequence between nucleotides 1 and 991 for detection of Gag RNA.

### Protocol:

Isolation of RNA from Prostate Tissue

The standard RNA isolation method from prostate tissues using Trizol reagent following manufacturer's instruction was used. The required amount of Trizol was added to a clean Petri-dish and the frozen tissue was minced directly in the reagent using disposable forceps. The yield was about 15-20 ugs RNA from <1 cm³ prostate tissue.

### Isolation of RNA in Expressed Prostate Secretion (EPS) and Urine

The prostatic fluids were collected in RNAse-free microfuge tubes by manually milking secretions from the prostate after the prostate was removed during surgery, flash frozen and stored at −80° C. until RNA isolation. The RNA isolation from 100-200 μl samples was performed using MagMAX™ Viral RNA Isolation kit (Ambion, Tex., USA) with some modifications as stated. For each isolation, the EPS sample was added to 602 μl of Lysis/Binding solution containing 300 μl of Lysis/Binding solution concentrate, 2 μl of carrier RNA and 300 μl of isopropanol. This was followed by addition of 40 μl of Bead Mix containing 20 μl of RNA binding beads and 20 μl of Lysis/Binding enhancer. The washing step was performed following manufacturer's protocol and eluted in 40-60 μl of preheated elution buffer. Generally, the amount of RNA obtained was in the range of 50-100 ng/μl as assessed using NanoDrop™ ND 1000 Spectrophotometer (NanoDrop Technologies).

qRT/PCR using AgPath-ID™ Kit

In one aspect, a two primer probe combination in separate reactions was used for the detection of XMRV in RNA isolated from a patient sample.

1. The standard RNA and prostate RNA were thawed in two different ice buckets to avoid cross contamination during addition.

2. At least six different dilution of RNA (10 fold each dilution) in RNA storage solution was made.

3. All the reagents of AgPath-ID™ kit were thawed on ice except the enzyme).

4. A master mix without RNA was made.

5. In 96 well plate the following were added on ice:

| | | |
|---|---|---|
| 2X RT/PCR buffer: | 12.5 | ul |
| 50 uM Forward Oligo: | 0.45 | ul (900 nM) |
| 50 uM Reverse Oligo: | 0.45 | ul (900 nM) |
| 10 uM probe: | 0.625 | ul (250 nM) |
| Water from kit: | 4.975 | ul |
| 25X RT/PCR enzyme: | 1 | ul |
| **RNA: | 3-5 | ul (add last) |
| Total volume: | 25 | ul |

**Limit prostate RNA to 100-200 ngs. Volume should not be less than 3 ul.

While using MGB probe, 1.25 ul of the probes was used instead of individual primers and probe.

### Instrument Set Up:

Set up the instrument as recommended by the manufacturer.

Condition:

Stage 1, Rep=1

Step 1: 45° C. for 10 mins

Stage 2, Rep=1

Step 1: 95° C. for 10 mins

Stage 3, Rep=55 ***

Step 1: 95° C. for 0.15 min

Step 2: 58° C. for 1.0 min (Data Collection)

* * * Generally, the prostate RNA has very low copy of XMRV RNA with Ct value of >35.

### Data Analysis:

Save the data and transfer to lab computer. Transfer the data to Excel spreadsheet for analysis. The graph with the standard RNA should generate R2 value of >0.98 and slope ˜−3.3 to −3.5.

### Results

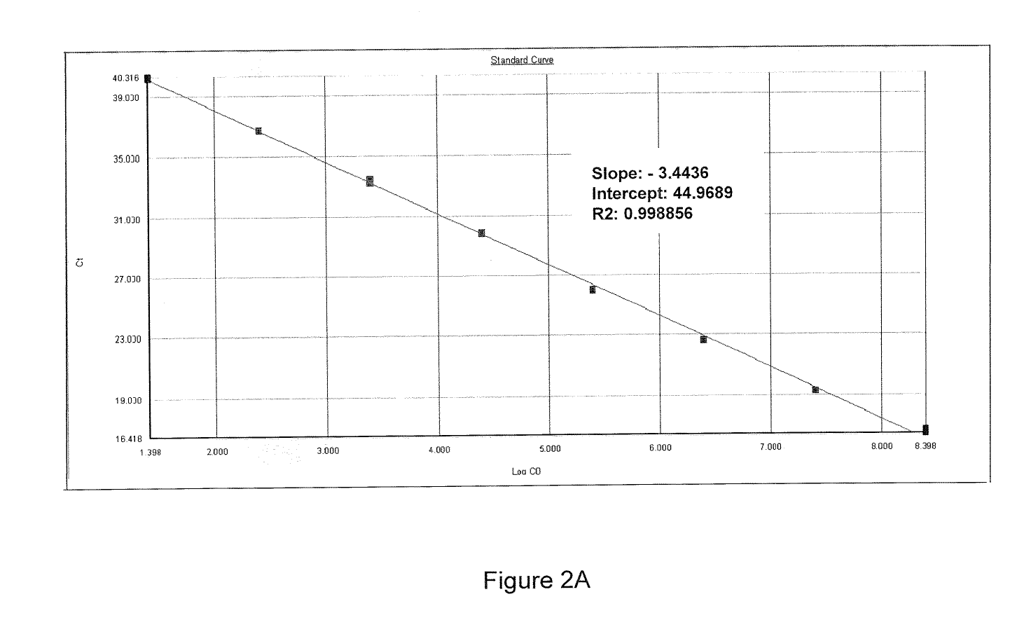

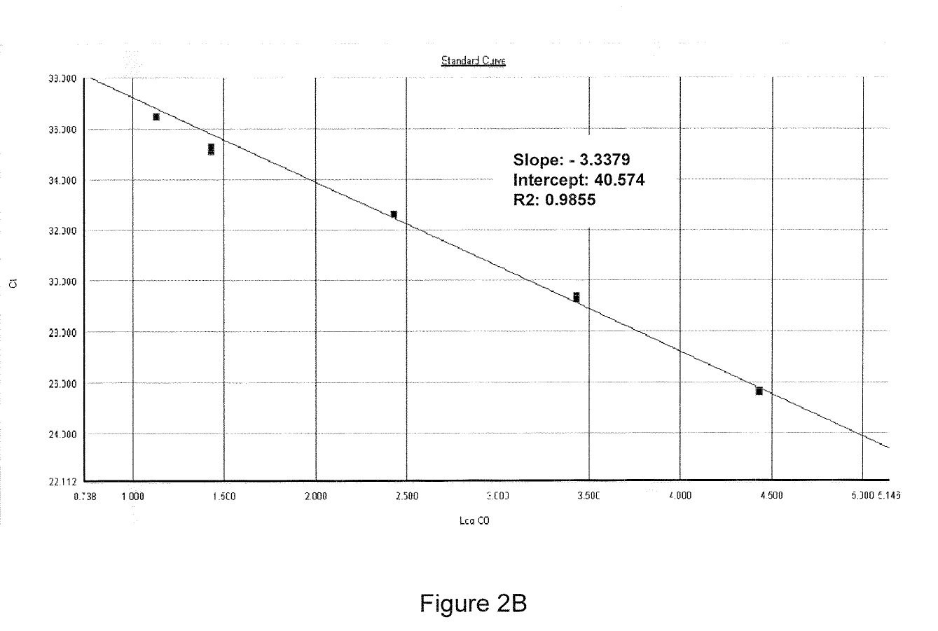

Standard gag and Envelope RNA was diluted to different dilution and performed qRT/PCR using Ag-Path kit. The standard curve results are shown in FIGS. 2A and 2B.

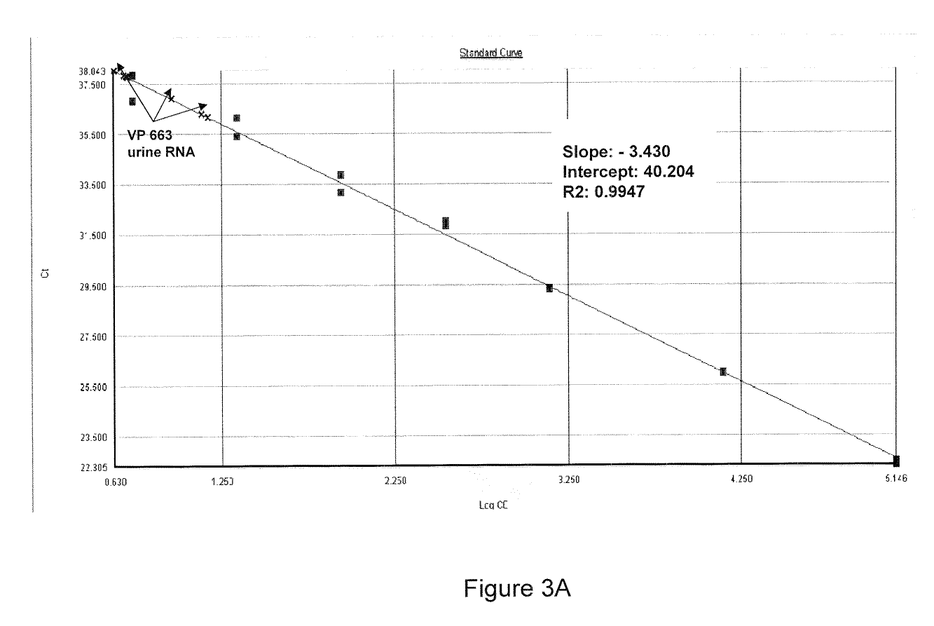

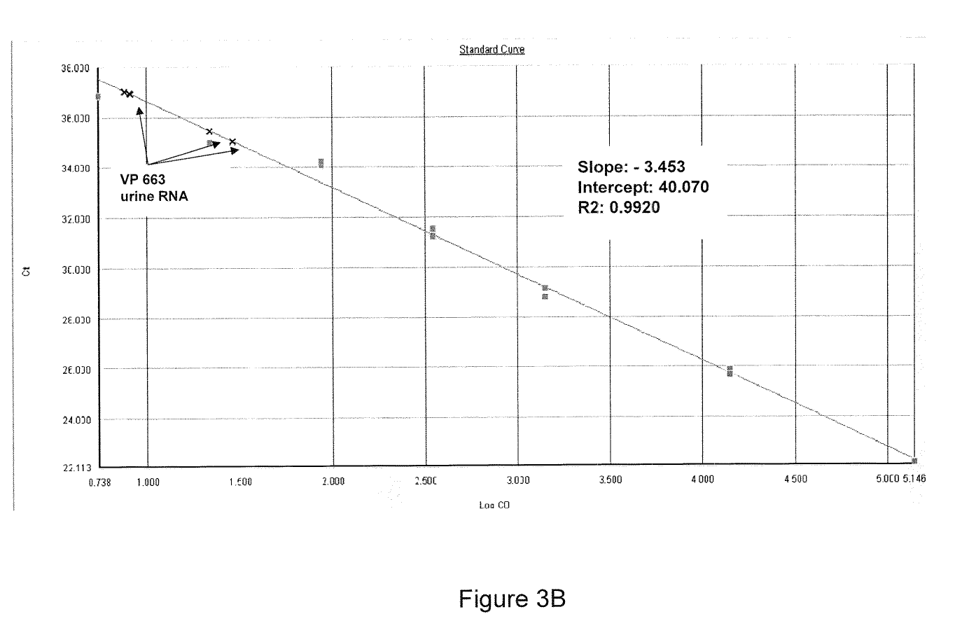

FIGS. 3A and 3B shows the results of XMRV RNA copy number in urine of prostate cancer patient VP663 using E1 and E2 sites in env, respectively. Results of qRT-PCR assays were performed six times (x) and is shown in comparison to a standard curve generated with a 1.85 kb XMRV env RNA produced by in vitro transcription. Y axis shows the Ct values, x axis shows the log of the copy number.

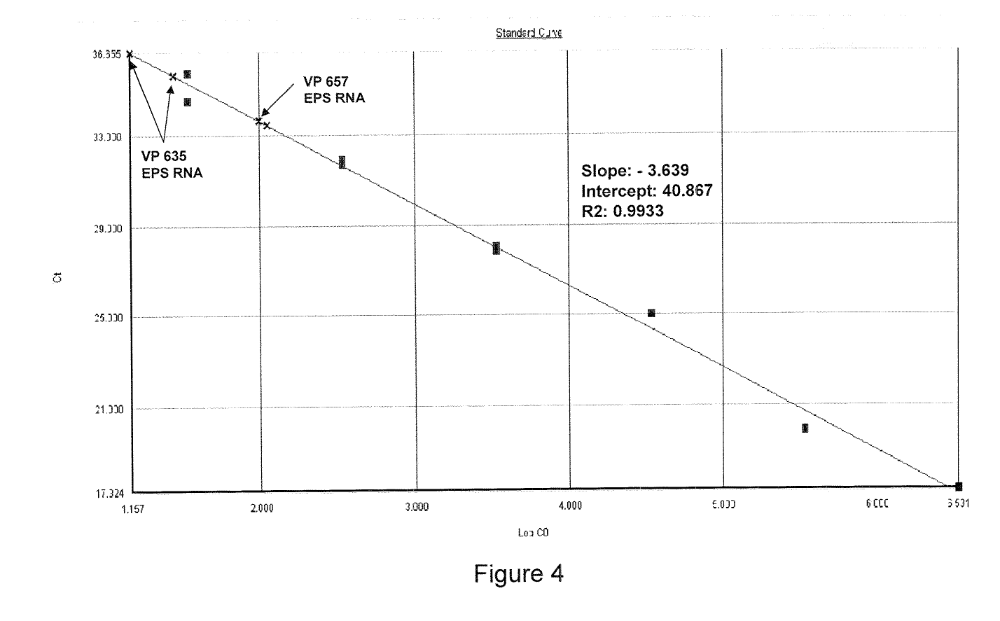

FIG. 4 shows qRT/PCR identification of XMRV RNA in prostate cancer patients (VP 635 and VP 657) expressed prostate secretions (EPS) by manual milking of the prostate during radical prostatectomy. Assays were done in duplicate as shown in comparison to a standard curve generated with a 1.85 kb XMRV env RNA produced by in vitro transcription. Y axis shows the Ct values, x axis shows that log of the copy number.

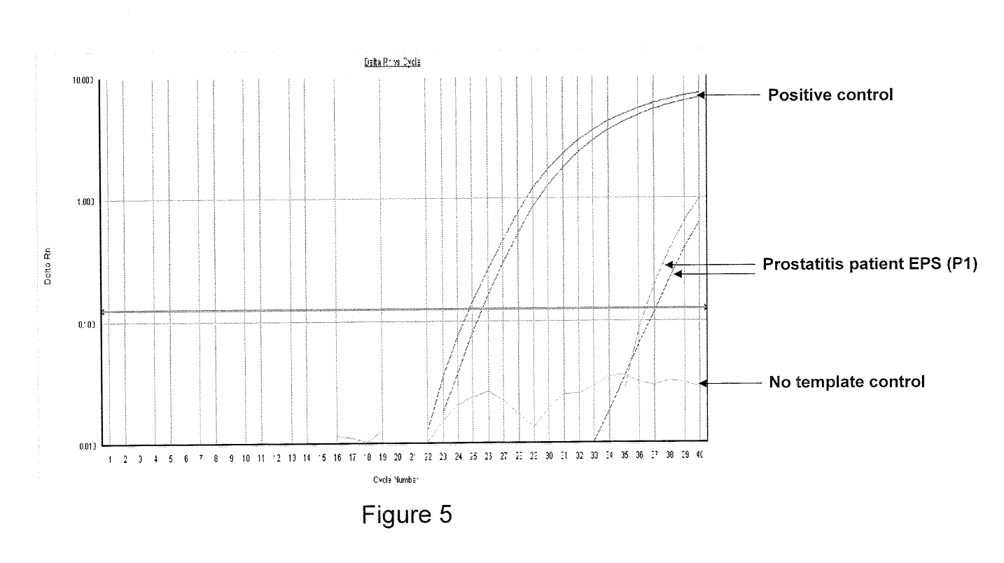

FIG. 5 shows the amplification plot of qRT-PCR analysis of XMRV RNA isolated from prostatitis patient's EPS (Patient P1). Very high Ct corresponds to low copy of XMRV RNA in the sample. In no template control sample, water was added in the reaction instead of RNA.

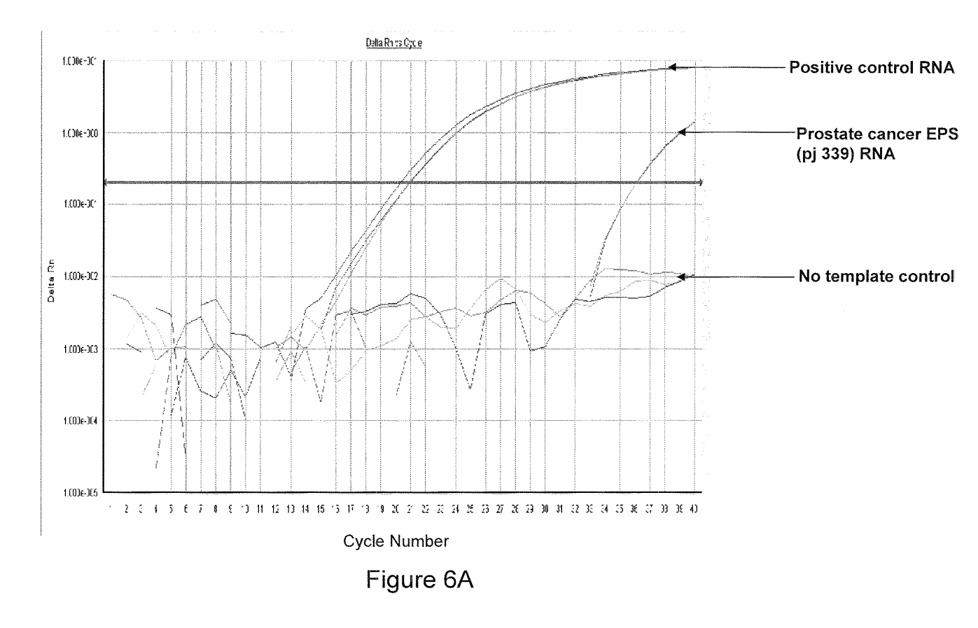

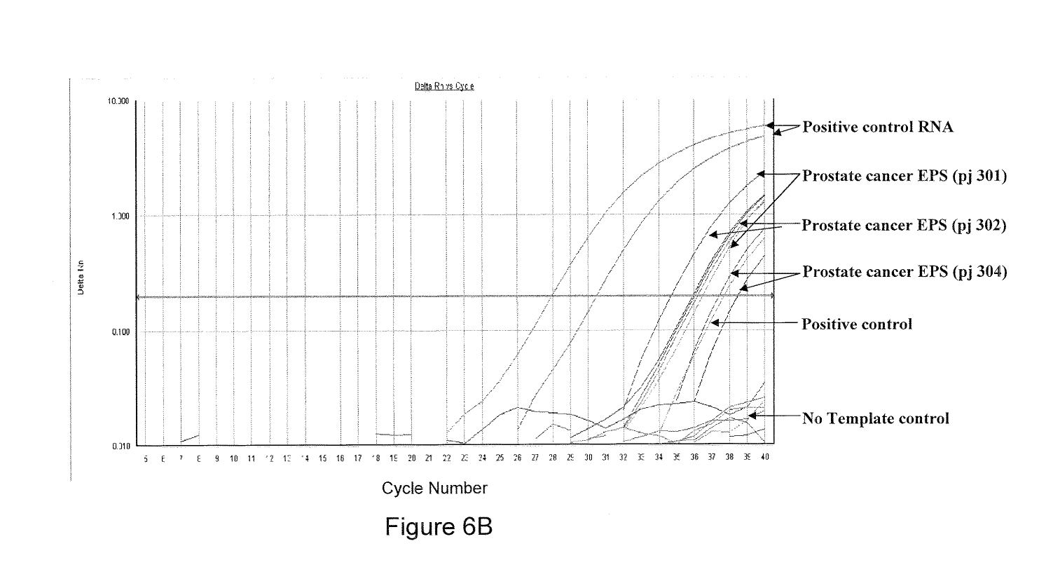

FIGS. 6A and 6B show the amplification of plot qRT-PCR analysis of XMRV RNA isolated from prostate cancer patients' EPS (pj 339, pj 301, pj 302, pj 304). Only one dilution of three positive standard RNA was used in the reaction.

### Example 2

### Detection of XMRV DNA by qPCR

### Reagents

QIAamp DNA mini kit (Qiagen Cat: 51306)

### TaqMan® Universal PCR Master Mix (Applied Biosystems Cat:430-4437)

### Protocol:

Using sterile forceps and scalpel, a slice of frozen prostate tissue was cut. The tissue slice was minced on a petri dish. DNA was isolated using a standard protocol from the QIAamp DNA mini kit. The DNA was alcohol precipitated, washed with 70% ethanol and resuspended in 20 μl of TE. About 250-500 ngs of DNA was used in each reaction.

### Instrument Set Up:

The instrument was set up as recommended by the manufacturer

Condition:

Stage 1, Rep=1

Step 1: 95° C. for 10 min

Stage 2, Rep=55 * * *

Step 1: 95° C. for 0.15 min

Step 2: 58° C. for 1.0 min (Data Collection)

FIG. 13 shows the qPCR generated amplification plot using DNA from prostate cancer patients VP 222, VP432 and VP 229. G1 primer probe combination was used in the reaction.

### Example 3

### Tth-Nested RT/PCR for Detection of XMRV RNA

### Recommendations

1. A clean area of the lab dedicated for patient samples only was assigned. High copy XMRV nucleic acids should not be present in this area.

2. During nested PCR, extreme precaution was taken to avoid cross-contamination from positive samples. It is advisable to standardize the PCR of positive control before the experimental sample. Use the condition for patient sample without any positive control.

3. Preferably separate pipettes were used to add reagents, which were not used to pipette high copy standard plasmid or RNA.

Standard RNA and PCR precautions were taken (e.g., powder free gloves, filter tips and clean area for RNA and PCR work).

### Materials:

### Oligonucleotides:

### Set 1:

### Round 1:

(SEQ ID NO: 22)

| | |
|---|---|
| 6200R: | CCCATGATGATGATGGCTTCCAGTATGC (20 μM) |

(SEQ ID NO: 23)

| | |
|---|---|
| 5922F: | GCTAATGCTACCTCCCTCCTGG (20 μM) |

(SEQ ID NO: 24)

| | |
|---|---|
| 350F: | GAGTTCGTATTCCCGGCCGCAGC (20 μM) |

(SEQ ID NO: 25)

| | |
|---|---|
| 718R: | GGTAACCCAGCGCCTCTTCTTGACATCC (20 μM) |

### Round 2:

| | |
|---|---|
| 5942F: | |
| GGGGACGATGACAGACACTTTCC (10 μM) | (SEQ ID NO: 26) |
| 6159R: | |
| CACATCCCCATTTGCCACAGTAG (10 μM) | (SEQ ID NO: 27) |
| 424F: | |
| ATCAGTTAACCTACCCGAGTCGGAC (10 μM) | (SEQ ID NO: 28) |
| 535R: | |
| GGTTTCGGCGTAAAACCGAAAGC (10 μM) | (SEQ ID NO: 29) |

### Set 2:

1st round:

(SEQ ID NO: 30)

| | |
|---|---|
| 6273R: | GGAGCCCACTGAGGAATCAAAACAGG (20 μM) |

(SEQ ID NO: 31)

| | |
|---|---|
| 5922F: | GCTAATGCTACCTCCCTCCTGG (20 μM) |

(SEQ ID NO: 32)

| | |
|---|---|
| 350F: | GAGTTCGTATTCCCGGCCGCAGC (20 μM) |

(SEQ ID NO: 33)

| | |
|---|---|
| 718R: | GGTAACCCAGCGCCTCTTCTTGACATCC (20 μM) |

2nd round:

(SEQ ID NO: 22)

| | |
|---|---|
| 6200R: | CCCATGATGATGATGGCTTCCAGTATGC (10 μM) |

(SEQ ID NO: 26)

| | |
|---|---|
| 5942F: | GGGGACGATGACAGACACTTTCC (10 μM) |

(SEQ ID NO: 28)

| | |
|---|---|
| 424F: | ATCAGTTAACCTACCCGAGTCGGAC (10 μM) |

(SEQ ID NO: 29)

| | |
|---|---|
| 535R: | GGTTTCGGCGTAAAACCGAAAGC (10 μM) |

The oligonucleotides were resuspended in 1×TE buffer.

### Reagents:

1×TE buffer (Cat: 75893. USB Corporation, Cleveland, Ohio, USA) DEPC treated RNase free water (Cat: 70783. USB Corporation, Cleveland, Ohio, USA)

PCR-Qualified Water (Cat: 71785. USB Corporation, Cleveland, Ohio, USA)

Tth DNA Polymerase kit (Cat: 70052. USB Corporation, Cleveland, Ohio, USA)

HotStart-IT· FideliTaq™ Master Mix 2× (Cat: 71156. USB Corporation, Cleveland, Ohio, USA)

PCR Nucleotide mix (Cat: 77212. USB Corporation, Cleveland, Ohio, USA)

RNA Storage solution (Ambion Cat: AM 7000)

RNase Inhibitor 40 u/ul (Cat: 71571. USB Corporation, Cleveland, Ohio, USA)

### RNA:

RNA was isolated from prostate cancer patient urine samples.

RNA was isolated from expressed prostate secretion (EPS) of prostate cancer patient during prostatectomy.

Positive control full length XMRV RNA was isolated from XMRV infected prostate cancer cell line.

### Method:

1. All the reagents were thawed on ice. The RNA was saved on dry ice until the reaction mixture was ready.
2. In sterile 200 μl PCR tubes on ice, the following were added:

| | | |
|---|---|---|
| RNA: | ~200-300 | ngs |
| 20 μM 6200R: | 1.5 | μl |
| 20 μM 718R: | 1.5 | μl |
| 10 mM dNTP: | 0.4 | μl |

| | |
|---|---|
| Water: | Up to 14 μl |

3. The tubes were placed at 70° C. for 5 minutes, and immediately chilled on ice for at least 1 minute. Then to individual tubes, the following were added:

| | |
|---|---|
| 5X buffer: | 2 μl (0.5X final) |
| MnCl₂: | 2 μl |
| Tth: | 1 μl |
| RNase Inhibitor: | 1 μl |

The tubes were placed on benchtop (−25° C.) for 5 minutes followed by incubation at 57° C. for 30 minutes.

4. The Chelate buffer was made by adding the following reagents on ice:

| | | |
|---|---|---|
| 5X Chelate buffer: | 20 | μl (From Tth Polymerase kit) |
| 20 μM 5922F: | 1.5 | μl |
| 20 μM 350F: | 1.5 | μl |
| 10 mM dNTP: | 3 | μl |
| PCR Qualified Water: | 50 | μl |
| Total: | 80 | μl |

5. After the reverse transcription step, 80 μl of chelate buffer was added to each tube and mixed properly.
6. The following program in the PCR machine was performed:
Step 1: 94° C. for 2 minutes
Step 2: 94° C. for 30 seconds
Step 3: 57° C. for 30 seconds
Step 4: 72° C. for 45 seconds
Step 5: G0 to step 2 for 45×
Step 6: 72° C. for 2 minutes
Step 7: 4° C. hold

### 7. 2nd Round PCR

Set up second round PCR mix by adding the following:

| | |
|---|---|
| 2X Hot Start master mix: | 25 μl |
| 10 μM 5942F: | 1 μl |
| 10 μM 6159R: | 1 μl |
| 10 μM 424F: | 1 μl |
| 10 μM 535R: | 1 μl |
| 25 mM MgCl₂: | 2 μl |
| 1st round PCR product: | 3 μl |
| PCR qualified water: | Up to 50 μl |

The following program in the PCR machine was performed:
Step 1: 94° C. for 2 minutes
Step 2: 94° C. for 30 seconds
Step 3: 57° C. for 30 seconds
Step 4: 72° C. for 45 seconds
Step 5: G0 to step 2 for 35×
Step 6: 72° C. for 2 minutes
Step: 7: 4° C. hold

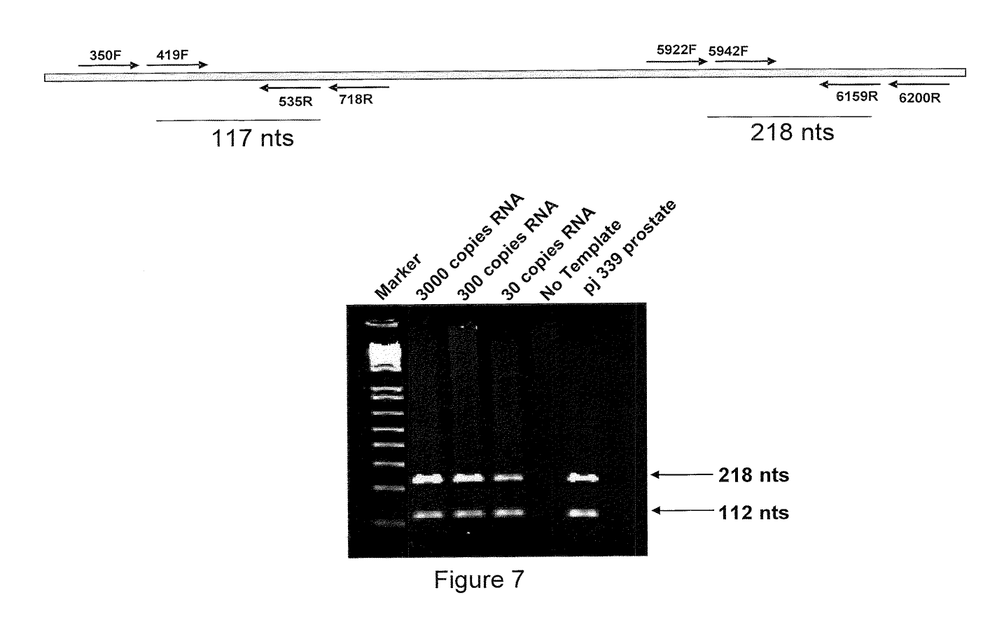

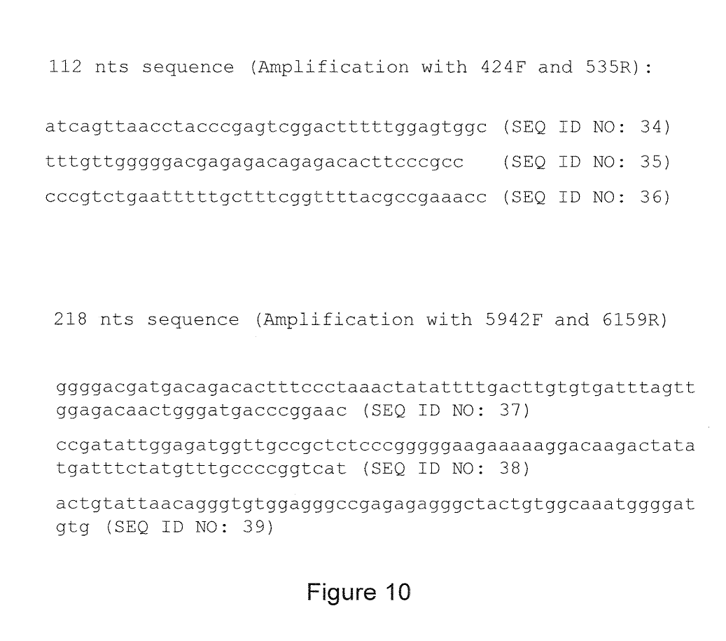

After the PCR, 8 ul of the product was loaded on to 2% Agarose gel and the band was visualized under UV transilluminator. A positive RNA generated bands of 218 and 112 nucleotides in length. In the upper panel of FIG. 7, the location of the primers used for multiplex RT-PCR of XMRV is shown. In the lower panel of FIG. 7, a gel of multiplex RT-PCR, which was performed using 3000 to 30 copies of XMRV RNA along with RNA isolated from a prostate cancer patient EPS (pj339), is shown. The respective sequences are shown in FIG. 10.

In one aspect, the PCR products are gel purified and the sequence is verified.

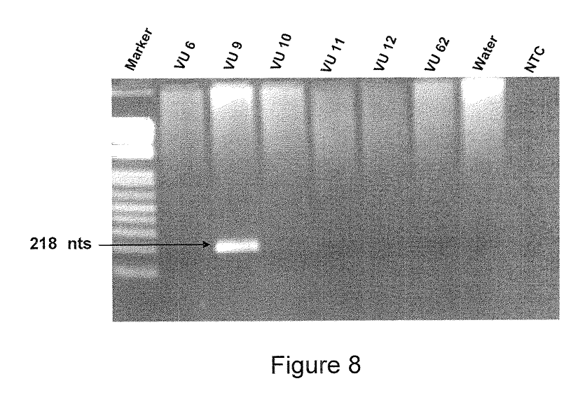

FIG. 8 is a gel of singleplex nested RT-PCR of RNA isolated from 6 prostate cancer patient urine samples using Tth and HotStart Polymerase following the protocol described above. Oligos 6200R and 5922F were used for the first round followed by 6159R and 5942F for the second round of amplification.

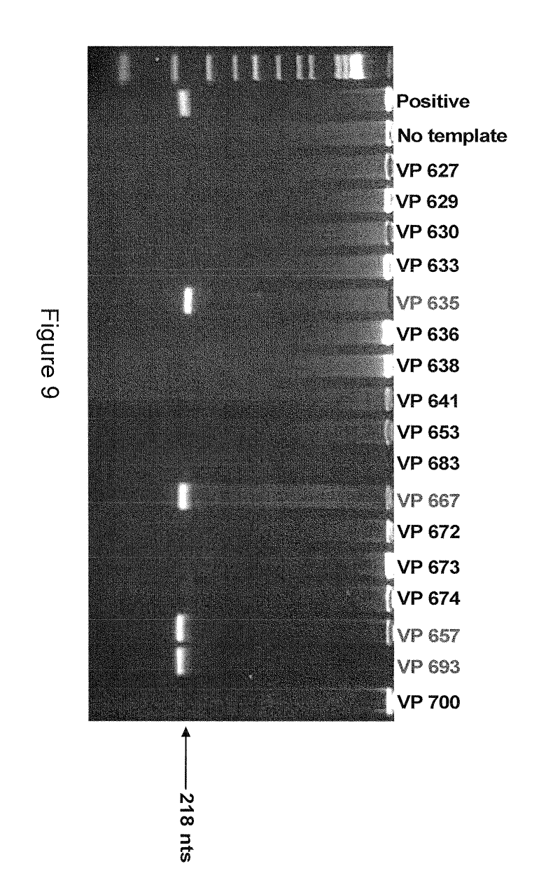

FIG. 9 is a gel of singleplex nested RT-PCR of RNA isolated from 17 prostate cancer patient expressed secretions during prostatectomy. Oligos 6200R and 5922F were used for the first round followed by 6159R and 5942F for the second round of amplification.

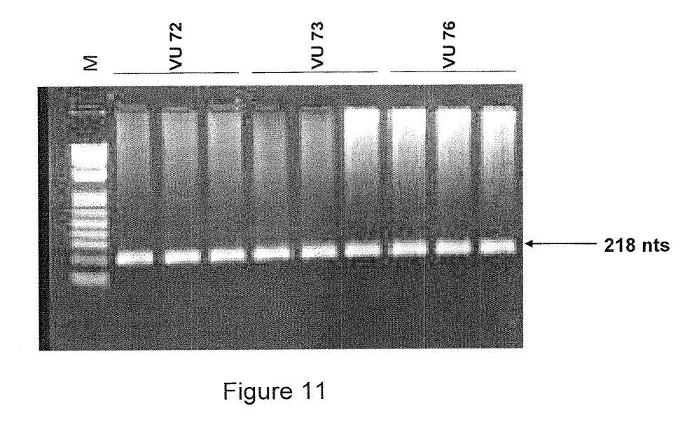

FIG. 11 is a gel of singleplex nested RT-PCR of RNA isolated from 3 prostate cancer patient urine samples, reaction time were done in triplicates. Oligos 6200R and 5922F were used for the first round followed by 6159R and 5942F for the second round of amplification.

### SUMMARY

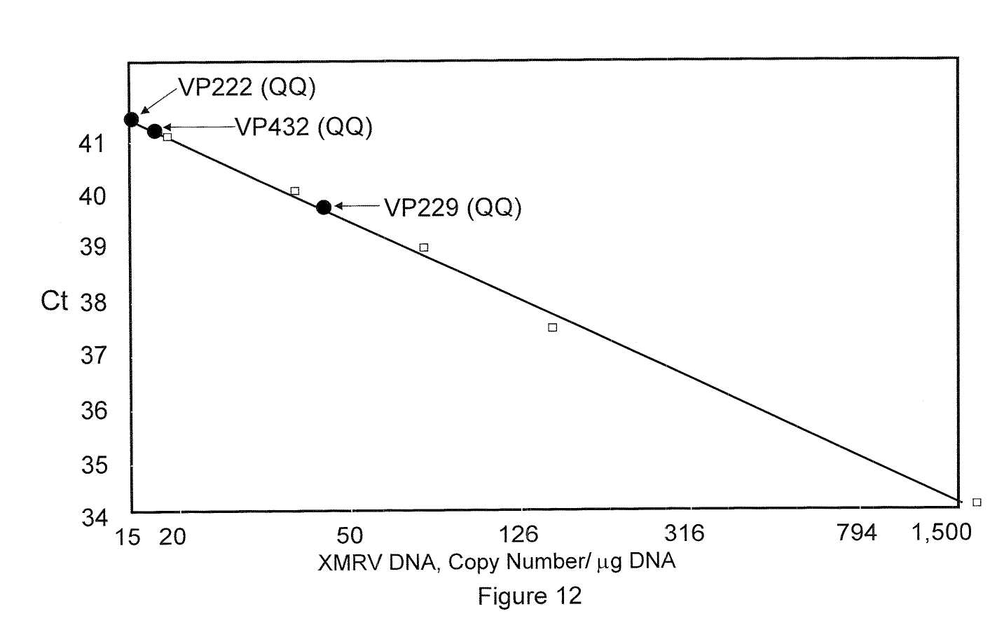

Prostate tissue, urine and prostatic secretions were collected from patients. For patients with prostate cancer, urine samples were obtained immediately prior to surgery. Prostatic fluid was also collected from the same patients at the time of prostatectomy by manually milking secretions from the prostate and seminal vesicles once the specimen had been removed from the patient. Approximately 50 prostate secretions and a similar number of urine samples from men with prostate cancer were assayed. About 20 bladder cancer tissue samples were also assayed and none were positive for XMRV. Regions of two XMRV genes (gag and env) were assayed in duplicate or triplicate. Evidence of XMRV in the prostatic tissue, prostatic secretions and urine of several men with prostate cancer has been demonstrated by simultaneously detecting XMRV gag and env sequences through qRT-PCR. A subset of these samples were confirmed by sequencing of the amplified regions of qRT/PCR respectively. In 5 cases, XMRV gag sequences isolated from the patient's urine and prostatic fluid were sequenced following PCR and found to be 100% identical to each other. The gag fragment also shared 100% homology with that of two XMRV strains, VP62 (GenBank Accession No. DQ399707) and VP35 (GenBank Accession No. DQ241301), and shared 98% homology with XMRV VP42 (GenBank Accession No. DQ241302). All three were from men with the QQ RNASEL genotype. In five cases a 218 nt region of the XMRV env gene was also identified by nested RT-PCR with a sequence that was identical with that of previously published strains for XMRV. An example of the detection of XMRV RNA by qRT-PCR in urine from a QQ RNASEL prostate cancer patient is presented (FIG. 3A). An example of a nested XMRV env RT-PCR product from a prostatic secretion isolated from a prostate cancer case with the RNASEL QQ genotype is shown (FIG. 3A). Detection and determination of XMRV DNA copy numbers were also determined in DNA isolated from tumor-bearing prostate tissues of men with the RNASEL QQ genotype following prostatectomy (Example 2, FIG. 12).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of detecting the presence of xenotropic MLV related virus (XMRV) in an individual comprising:
a) contacting a sample of the individual with at least one set of primers wherein the set of primers comprises:
at least one forward primer and at least one reverse primer which are complementary to all or a portion of an XMRV G1 gag nucleotide sequence,
at least one forward primer and at least one reverse primer which are complementary to all or a portion of an XMRV G2 gag nucleotide sequence,
at least one forward primer and at least one reverse primer which are complementary to all or a portion of an XMRV G3 gag nucleotide sequence,
at least one forward primer and at least one reverse primer which are complementary to all or a portion of an XMRV E1 envelope nucleotide sequence,
at least one forward primer and at least one reverse primer which are complementary to all or a portion of an XMRV E2 envelope nucleotide sequence,
at least one forward primer and at least one reverse primer which are complementary to all or a portion of an XMRV E3 envelope nucleotide sequence,
at least one forward primer and at least one reverse primer which are complementary to all or a portion of an XMRV P1 Pol nucleotide sequence,
or a combination thereof;
b) maintaining the sample under conditions which amplify the primers if XMRV is present in the sample to produce amplified XMRV sequences;
c) detecting whether amplified XMRV sequences are present in the sample;
wherein if amplified XMRV sequences are detected in the sample, then XMRV is present in the individual.

2. The method of claim 1 wherein the amplified XMRV sequences are amplified XMRV DNA sequences, XMRV RNA sequences or a combination thereof.

3. The method of claim 1 wherein a polymerase chain reaction (PCR) is used to amplify the primers.

4. The method of claim 3 wherein the PCR is real time PCR.

5. The method of claim 4 further comprising contacting the sample with one or more fluorescently labeled probes.

6. The method of claim 5 wherein the probe is labeled with fluorescein.

7. The method of claim 5 wherein the forward primer complementary to all or a portion of an XMRV G1 gag nucleotide sequence has a nucleotide sequence comprising GGACTTTTTGGAGTGGCTTTGTT (SEQ ID NO: 1), the reverse primer complementary to all or a portion of an XMRV G1 gag nucleotide sequence has a nucleotide sequence comprising GCGTAAAACCGAAAGCAAAAT (SEQ ID NO: 2) and the probe has a nucleotide sequence comprising ACAGAGACACTTCCCGCCCCCG (SEQ ID NO: 3).

8. The method of claim 5 wherein the forward primer complementary to all or a portion of an XMRV G2 gag nucleotide sequence has a nucleotide sequence comprising GTAACTACCCCTCTGAGTCTAACCT (SEQ ID NO: 4), the reverse primer complementary to all or a portion of an XMRV G3 gag nucleotide sequence has a nucleotide sequence comprising CTTCTTGACATCCACAGACTGGTT (SEQ ID NO: 5) and the probe has a nucleotide sequence comprising TCCAGCGCATTGCATC (SEQ ID NO: 6).

9. The method of claim 5 wherein the forward primer complementary to all or a portion of an XMRV G3 gag nucleotide sequence has a nucleotide sequence comprising CTCAGGTCAAGTCTAGAGTGTTTTGT (SEQ ID NO: 7), the reverse primer complementary to all or a portion of an XMRV G2 gag nucleotide sequence has a nucleotide sequence comprising CCTCCCAGGTGACGATATATGG (SEQ ID NO: 8) and the probe has a nucleotide sequence comprising CCCCACGGACACCC (SEQ ID NO: 9).

10. The method of claim 5 wherein the forward primer complementary to all or a portion of an XMRV P1 pol nucleotide sequence has a nucleotide sequence comprising CGGGACAGAACTATCCAGTATGTGA (SEQ ID NO: 10), the reverse primer complementary to all or a portion of an XMRV P1 pol nucleotide sequence has a nucleotide sequence comprising TGGCTTTGCTGGCATTTACTTG (SEQ ID NO: 11) and the probe has a nucleotide sequence comprising ACCTGCACCGCCTGTG (SEQ ID NO: 12).

11. The method of claim 5 wherein the forward primer complementary to all or a portion of an XMRV E1 env nucleotide sequence has a nucleotide sequence comprising GGCCGAGAGAGGGCTACT (SEQ ID NO: 13), the reverse primer complementary to all or a portion of an XMRV E1 env nucleotide sequence has a nucleotide sequence comprising TGATGATGATGGCTTCCAGTATGC (SEQ ID NO: 14) and the probe has a nucleotide sequence comprising CACATCCCCATTTGCC (SEQ ID NO: 15).

12. The method of claim 5 wherein the forward primer complementary to all or a portion of an XMRV E2 env nucleotide sequence has a nucleotide sequence comprising CCCTAGTGGCCACCAAACAA (SEQ ID NO: 16), the reverse primer complementary to all or a portion of an XMRV E2 env nucleotide sequence has a nucleotide sequence comprising AAGGCCCCAAGGTCTGTATGT (SEQ ID NO: 17) and the probe has a nucleotide sequence comprising TCGAGCAGCTCCAGGCAGCCA (SEQ ID NO: 18).

13. The method of claim 5 wherein the forward primer complementary to all or a portion of an XMRV E3 env nucleotide sequence has a nucleotide sequence comprising TCAGGACAAGGGTGGTTTGAG (SEQ ID NO: 19), the reverse primer complementary to all or a portion of an XMRV E3 env nucleotide sequence has a nucleotide sequence comprising GGCCCATAATGGTGGATATCA (SEQ ID NO: 20) and the probe has a nucleotide sequence comprising TTAACAGGTCCCCATGGTTCACGACCA (SEQ ID NO: 21).

14. The method of claim 3 wherein the PCR is nested, reverse transcription PCR comprising a first round of PCR and a second round of PCR.

15. The method of claim 14 wherein
in the first round of PCR, the forward primer for the gag sequence has a nucleotide sequence comprising GAGTTCGTATTCCCGGCCGCAGC (SEQ ID NO: 24), the reverse primer for the gag sequence has a nucleotide sequence comprising GGTAACCCAGCGCCTCTTCTTGACATCC (SEQ ID NO: 25), the forward primer for the env sequence has a nucleotide sequence comprising CCCATGATGATGATGGCTTCCAGTATGC (SEQ ID NO: 22) and the reverse primer for the env sequence has a nucleotide sequence comprising GCTAATGCTACCTCCCTCCTGG (SEQ ID NO: 23); and
in the second round of PCR, the forward primer for the gag sequence has a nucleotide sequence comprising ATCAGTTAACCTACCCGAGTCGGAC (SEQ ID NO: 28), the reverse primer for the gag sequence has a nucleotide sequence comprising GGTTTCGGCGTAAAACCGAAAGC (SEQ ID NO: 29), the forward primer for the env sequence has a nucleotide sequence comprising GGGGACGATGACAGACACTTTCC (SEQ ID NO: 26) and the reverse primer for the env sequence has a nucleotide sequence comprising CACATCCCCATTTGCCACAGTAG (SEQ ID NO: 27).

16. The method of claim 1 wherein the amplified XMRV sequences are detected using gel electrophoresis.

17. The method of claim 1 further comprising cloning the amplified XMRV sequences.

18. The method of claim 1 further comprising comparing the amplified XMRV sequences to a control.

19. The method of claim 1 wherein the sample is selected from the group consisting of: urine, prostate tissue, prostatic fluids, bladder cancer tissue or a combination thereof.

20. The method of claim 19 wherein the prostatic fluid is an expressed prostate secretion (EPS).

21. The method of claim 20 wherein the EPS is semen.

22. The method of claim 1 wherein the individual is a human.

23. A method of detecting prostate cancer at an early stage in an individual comprising:
a) contacting a sample of the individual with at least one set of primers wherein the set of primers comprises:
at least one forward primer and at least one reverse primer which are complementary to all or a portion of an XMRV G1 gag nucleotide sequence,
at least one forward primer and at least one reverse primer which are complementary to all or a portion of an XMRV G2 gag nucleotide sequence,
at least one forward primer and at least one reverse primer which are complementary to all or a portion of an XMRV G3 gag nucleotide sequence,
at least one forward primer and at least one reverse primer which are complementary to all or a portion of an XMRV E1 envelope nucleotide sequence,
at least one forward primer and at least one reverse primer which are complementary to all or a portion of an XMRV E2 envelope nucleotide sequence,
at least one forward primer and at least one reverse primer which are complementary to all or a portion of an XMRV E3 envelope nucleotide sequence,
at least one forward primer and at least one reverse primer which are complementary to all or a portion of an XMRV P1 Pol nucleotide sequence,
or a combination thereof;
b) maintaining the sample under conditions which amplify the primers if XMRV is present in the sample to produce amplified XMRV sequences;
c) detecting whether amplified XMRV sequences are present in the sample;
wherein the detection of amplified XMRV sequences in the sample indicates that the individual has prostate cancer at an early stage.

24. A method of detecting an individual at risk for developing prostate cancer comprising:
a) contacting a sample of the individual with at least one set of primers wherein the set of primers comprises:
at least one forward primer and at least one reverse primer which are complementary to all or a portion of an XMRV G1 gag nucleotide sequence,
at least one forward primer and at least one reverse primer which are complementary to all or a portion of an XMRV G2 gag nucleotide sequence,
at least one forward primer and at least one reverse primer which are complementary to all or a portion of an XMRV G3 gag nucleotide sequence,
at least one forward primer and at least one reverse primer which are complementary to all or a portion of an XMRV E1 envelope nucleotide sequence,
at least one forward primer and at least one reverse primer which are complementary to all or a portion of an XMRV E2 envelope nucleotide sequence,
at least one forward primer and at least one reverse primer which are complementary to all or a portion of an XMRV E3 envelope nucleotide sequence,
at least one forward primer and at least one reverse primer which are complementary to all or a portion of an XMRV P1 Pol nucleotide sequence,
or a combination thereof;
b) maintaining the sample under conditions which amplify the primers if XMRV is present in the sample to produce amplified XMRV sequences;
c) detecting whether amplified XMRV sequences are present in the sample;
wherein the detection of amplified XMRV sequences in the sample indicates that the individual is at risk for developing prostate cancer.

25. A method of detecting recurrence of prostate cancer in an individual comprising:
a) contacting a sample of the individual with at least one set of primers wherein the set of primers comprises:
at least one forward primer and at least one reverse primer which are complementary to all or a portion of an XMRV G1 gag nucleotide sequence,
at least one forward primer and at least one reverse primer which are complementary to all or a portion of an XMRV G2 gag nucleotide sequence,
at least one forward primer and at least one reverse primer which are complementary to all or a portion of an XMRV G3 gag nucleotide sequence,
at least one forward primer and at least one reverse primer which are complementary to all or a portion of an XMRV E1 envelope nucleotide sequence,
at least one forward primer and at least one reverse primer which are complementary to all or a portion of an XMRV E2 envelope nucleotide sequence,
at least one forward primer and at least one reverse primer which are complementary to all or a portion of an XMRV E3 envelope nucleotide sequence,
at least one forward primer and at least one reverse primer which are complementary to all or a portion of an XMRV P1 Pol nucleotide sequence,
or a combination thereof;
b) maintaining the sample under conditions which amplify the primers if XMRV is present in the sample to produce amplified XMRV sequences;
c) detecting whether amplified XMRV sequences are present in the sample;
wherein the detection of amplified XMRV sequences in the sample indicates the recurrence of prostate cancer in the individual.

26. A method of monitoring a treatment of an individual that has prostate cancer comprising:
a) contacting a sample of the individual with at least one set of primers wherein the set of primers comprises:
at least one forward primer and at least one reverse primer which are complementary to all or a portion of an XMRV G1 gag nucleotide sequence,
at least one forward primer and at least one reverse primer which are complementary to all or a portion of an XMRV G2 gag nucleotide sequence,
at least one forward primer and at least one reverse primer which are complementary to all or a portion of an XMRV G3 gag nucleotide sequence,
at least one forward primer and at least one reverse primer which are complementary to all or a portion of an XMRV E1 envelope nucleotide sequence,
at least one forward primer and at least one reverse primer which are complementary to all or a portion of an XMRV E2 envelope nucleotide sequence,
at least one forward primer and at least one reverse primer which are complementary to all or a portion of an XMRV E3 envelope nucleotide sequence,
at least one forward primer and at least one reverse primer which are complementary to all or a portion of an XMRV P1 Pol nucleotide sequence;
or a combination thereof;
b) maintaining the sample under conditions which amplify the primers if XMRV is present in the sample to produce amplified XMRV sequences;
c) detecting whether amplified XMRV sequences are present in the sample.
